(19) United States
(12) Patent Application Publication
(10) Pub. No.: US 2025/0270496 A1
(43) Pub. Date: Aug. 28, 2025

(54) PRODUCTION OF FUNGAL BIOMASS

(71) Applicant: MUSHLABS GMBH, Hamburg (DE)

(72) Inventors: Thibault Godard, Hamburg (DE); Daniel Alejandro Delgado Montafur, Hamburg (DE); Guido Albanese, Hamburg (DE); Mazen Rizk, Hamburg (DE)

(73) Assignee: MUSHLABS GMBH, Hamburg (DE)

(21) Appl. No.: 18/255,420

(22) PCT Filed: Dec. 24, 2021
(86) PCT No.: PCT/EP2021/087661

§ 371 Date: Jun. 1, 2023

(30) Foreign Application Priority Data
Dec. 24, 2020 (EP) 20217228.4

(51) Int. Cl. C12N 1/14; A23J 1/00; A23L 31/00; C12N 1/22

(52) U.S. Cl. CPC C12N 1/145; A23J 1/008; A23L 31/00; C12N 1/22

(57) ABSTRACT

The present invention relates to a method for the production of a fungal fermentation medium from at least one lignocellulosic material, preferably at least one industrial and/or agricultural side stream and to a fermentation medium obtainable accordingly, to a method for production of a fungal biomass by submerged fermentation of at least one fungal strain and to a fungal biomass obtainable accordingly, and to a fungal-based food product obtainable by using the instant fungal biomass of the invention. The instant fermentation medium produced preferably from spent grain is particularly useful in production of fungal biomass by submerged fermentation of Pleurotus pulmonarius, among others.

### RELATED APPLICATIONS

This application is a 35 U.S.C. § 371 filing of International Patent Application No. PCT/EP2021/087661, filed Dec. 24, 2021, which claims priority to European Patent Application No. 20217228.4, filed Dec. 24, 2020, the entire disclosures of which are hereby incorporated herein by reference.

### FIELD OF THE INVENTION

The present invention relates to a method for the production of a fungal fermentation medium from at least one lignocellulosic material (e.g., industrial and/or agricultural side stream) and to a fermentation medium obtainable accordingly, to a method for production of a fungal biomass by submerged fermentation of at least one fungal strain and to a fungal biomass obtainable accordingly, and to a fungal-based food product obtainable by using the instant fungal biomass of the invention.

### BACKGROUND OF THE INVENTION

In recent years, production of food from animals has been receiving attention because of its unsustainability as well as rising concerns about animal welfare. In the context of climate change, many plant-based meat-alternatives have emerged with the aim to cut down CO₂ emissions and reduce animal suffering. However, these products are currently produced from three major monocrops (soy, pea and rice) whose culture requires a lot of land and water, heavily relies on chemical agents (pesticides and fertilizers) and generates a lot of wastes as only protein isolated from these crops is used for production of meat alternatives. In addition, these isolates have a strong bitter taste and no intrinsic texture and therefore their use in foods requires further processing steps as well as the addition of further ingredients, including but not limited to flavouring agents, texturizers, and/or colorants. Hence, plant-based alternatives are not necessarily healthy, and their production induces other environmental issues such as deforestation, significant reduction of biodiversity, soil pollution, and/or water contamination.

Production of food using fermentation processes seems to address several of these drawbacks. It enables a better use of land as fermenters can be scaled vertically and allows for the production of food locally in cities or villages. Moreover, they require less water per kilo product than plant protein, and with ongoing development and improvement of filtration and treatment technologies, this water could be recycled in the process. Herein disclosed is the production of fungal mycelium as new food product using a novel fermentation process wherein the growth medium as well as the final product are at least partially produced using lignocellulosic materials, e.g. industrial and/or agricultural sidestreams as raw material. In that sense, the process described herein contributes to the efforts to build a circular economy wherein industrial, food and agricultural wastes are reduced to a minimum and resources are used to their fullest extent. Another advantage of fungal fermentation over production of conventional plant isolates is comprised within the obtained raw material—fungal biomass—that per nature already has a desired fibrous texture and brings a balanced nutritional profile with complete proteins but also dietary fibres, vitamins and micronutrients that provide consumers with a healthy product. In particular, the use of mycelium isolated from the fruiting bodies from known edible mushrooms additionally brings a typical mushroom umami taste specific to this group, varying a bit between the species (e.g. morel, truffle or button mushroom) and enables the production of clean and tasty products with a very short list of ingredient.

DE10201410884 describes a process for deodorising lignin comprising the step of extracting a lignocellulosic substrate with a supercritical fluid or supercritical fluid mixture.

DE102016110653 relates to a food-product/fermentation product that comprises mycelia of fungi.

CN101838673A discloses the fermentation of a fungus of the Basidomycota family in a liquid fermentation media complemented with rice distiller grain.

CN1078872A discloses a method for the preparation of a drink comprising the cultivation of a fungus in a fermentation media comprising amongst other components vinasse.

WO 2017/208255A1 relates to a method of preparing edible fungi (of the phylum Ascomycota) by cultivation in media comprising vinasse.

WO2002090527A1 relates to a method of preparation of edible fungi (e.g. Fusarium species).

WO2017/181085A1 discloses certain methods for the production of fungal mycelia.

WO 2019/046480A1 relates to the preparation of edible filamentous fungal formulation by growing filamentous fungal biomats.

RU 2006/126554 relates to a method of producing food and feed biomass on nutrient media based on waste from distillery stillage production, which involves the sequential cultivation of baker's yeast Saccharomyces cerevisiae and edible basidiomycetes, for example, selected from the group including Pleurotus ostreatus, Pleurotus pulmonarius, among others.

U.S. Pat. No. 5,846,787 discloses a process for the treatment of cellulose containing material.

Papadaki (doi: 10.3390/microorganisms7070207) discloses the cultivation of Pleurotus species (P. pulmonarius and P. ostreatus) by solid state fermentation and semiliquid fermentation using grape pomace as sidestream.

Kim Min-Keun et al. (Korean Journal of Mycology, DOI: 10.4489/KJM.2012.40.1.049) discloses development of the optimal media for mycelial culture of Pleurotus eryngii using the hot water extract of raw materials. Described process is solid state fermentation for the production of fruiting bodies, wherein no steam is used.

Platt M. W. et al (Eur. J. Appl. Microbiol. Biotechnol vol. 17, pages 140-142, 1983) disclose increased degradation of straw by Pleurotus ostreatus sp. ‘florida’. Disclosed process is a solid state fermentation.

Beltran-Garcia M. J. et al. (Revista de la Sociedad Quimica de Mexico, vol 45, pages 77-81, 2001) disclose that lignin degradation products from corn stalks enhance notably the radial growth of basidiomycete mushroom mycelia.

Document CN 104 446 687 A discloses certain liquid culture medium for tremella aurantialba submerged fermentation. It is noted that the extract from rice straw as disclosed in this document does not comprise more than 4% of the final medium.

Document CN 108 203 693 A discloses certain Rhizopus oryzae seed culture medium.

Document KR 2013/0057507 discloses certain method of cultivation of Cordyceps militaris.

Document ES 2′370′215 discloses certain means of fungal cultivation.

Document U.S. Pat. No. 3,576,720 discloses certain process for the continuous production of torula yeast from coffee berry waste.

Sidana Arushdeep et al. (Chinese Journal of Biology, vol 2014, pages 1 to 5) disclose sugarcane bagasse as a potential medium for fungal cultures.

Document U.S. Pat. No. 9,206,446 discloses certain extraction method from plant biomass.

### SUMMARY OF THE INVENTION

Particularly desirable are means and methods that utilize lignocellulosic material, preferably agricultural and/or industrial waste, herein industrial and/or agricultural side stream(s), as they are cost effective and more sustainable. Further particularly desirable are methods that lead to obtaining the fungal biomass, and consequently the food product, with amino acid composition reflecting that of a complete protein according to FAO definition (www.fao.org, https://en.wikipedia.org/wiki/Complete_protein).

Further particularly desirable is a method for production of the fungal biomass that is resistant to contamination with other microorganisms, for example with bacteria. Accordingly, a fungal fermentation medium resistant to contamination with bacteria, obtainable by using lignocellulosic material, preferably an industrial and/or agricultural side stream(s) (i.e. waste products) is particularly desirable.

It was an object of the present invention to provide improved means and methods for the production of a fungal-based food product, methods for the production of a fungal fermentation medium from a lignocellulosic material, preferably from agricultural and/or industrial sidestream(s) as well as methods and means for the production of fungal biomass for the use in the production of fungal-based food products.

The problem described herein is solved by the embodiments described in the following and as characterized in the claims.

The invention is summarized in the following embodiments.

In one embodiment, the present invention relates to a method for the production of a fungal fermentation medium from at least one lignocellulosic material, preferably an industrial and/or agricultural side stream, the method comprising: (a) aqueous extraction of the at least one industrial and/or agricultural side stream; and (b) combination of the aqueous extract(s) obtained in (a) with optionally at least one nutrient supplement for fungal cultivation.

In a particular embodiment, the present invention relates to a method for the production of a fungal fermentation medium from at least one lignocellulosic material, preferably an industrial and/or agricultural side stream, wherein the step (a) includes the step of prehydrolysis with steam followed by washing step performed with liquid water.

In a further particular embodiment, the present invention relates to a method for the production of a fungal fermentation medium from at least one lignocellulosic material, preferably an industrial and/or agricultural side stream, wherein during the prehydrolysis with steam the lignocellulosic materials contacted with steam at the temperature of more than 100° C., preferably at the temperature of between 150° C. and 300° C., more preferably at the temperature of between 160° C. and 180° C., even more preferably at the temperature of about 170° C., for a time of up to 20 minutes, preferably for a time of between 5 and 15 minutes, more preferably for a time of between 7.5 and 15 minutes.

In a further particular embodiment, the present invention relates to a method for the production of a fungal fermentation medium from at least one lignocellulosic material, preferably an industrial and/or agricultural side stream, wherein the lignocellulosic material upon prehydrolysis with steam is washed with liquid water, preferably at the temperature of 50° C. to 100° C., more preferably at the temperature of 50° C. to 70° C., even more preferably at the temperature of 50° C. to 60° C.

In a further particular embodiment, the present invention relates to a method for the production of a fungal fermentation medium from at least one lignocellulosic material, preferably an industrial and/or agricultural side stream, wherein (a) is performed with liquid water at a pressure of between 10 and 220 bar and at a temperature of between 9° and 374° C. for a time of between 10 and 200 minutes.

In a further particular embodiment, the present invention relates to a method for the production of a fungal fermentation medium from at least one lignocellulosic material, preferably an industrial and/or agricultural side stream, wherein (a) is performed with water at a pH of between 2.0 and 12.0, preferably of between 3.0 and 10.0, more preferably of between 4.0 and 8.0, most preferably of between 5.0 and 8.0.

In again a further particular embodiment, the present invention relates to a method for the production of a fungal fermentation medium from at least one lignocellulosic material, preferably an industrial and/or agricultural side stream, further comprising steps of processing the aqueous extract(s) obtained in (a) before the step (b) as follows:
i. proteins are separated from the aqueous extract(s) preferably by flocculation or by precipitation with CO₂;
ii. optionally proteins obtained in i. are hydrolyzed, preferably by using proteolytic enzymes, in particular selected from alcalases, papain, proteinase K, and trypsin, at a concentration of between 0.01% and 5% (w/w) and/or at a temperature of between 15 and 100° C. and/or for a time of between 0.5 and 96 hours;
iii. C5-polysaccharides present in the product of i. are hydrolyzed optionally using hemicellulases to monosaccharides, in particular xylose and/or arabinose; and
iv. product(s) of steps ii. and/or iii. are further used in step (b).

In again a further particular embodiment, the present invention relates to a method for the production of a fungal fermentation medium from at least one lignocellulosic material, preferably an industrial and/or agricultural side stream, wherein (a) involves the steps of:
(a1) extraction of the industrial and/or agricultural side stream with water, optionally supplemented by NaOH at a concentration of between 0.1% to 1.0% w/w, at a temperature of between 90 to 374° C., preferably of between 10° and 220° C., more preferably of between 110 to 180° C., for a time of between 10 to 200 minutes; and
(a2) extraction of the industrial and/or agricultural side stream with water at a temperature of between 12° and 220° C., preferably of between 12° and 190° C., for a time of between 5 and 150 minutes.

In again a further particular embodiment, the present invention relates to a method for the production of a fungal fermentation medium from at least one lignocellulosic material, preferably an industrial and/or agricultural side stream, wherein proteins present in the aqueous extract obtained in (a1) are isolated preferably by flocculation or by precipitation with CO₂.

In again a further particular embodiment, the present invention relates to a method for the production of a fungal fermentation medium from at least one lignocellulosic material, preferably an industrial and/or agricultural side stream, further comprising a step wherein proteins present in the aqueous extract obtained in (a1) are hydrolyzed before the step (b).

In again a further particular embodiment, the present invention relates to a method for the production of a fungal fermentation medium from at least one lignocellulosic material, preferably an industrial and/or agricultural side stream, further comprising a step wherein C5-polysaccharides present in the aqueous extract obtained in (a2) are further hydrolyzed to monosaccharides optionally using hemicellulases before the step (b).

In again a further particular embodiment, the present invention relates to a method for the production of a fungal fermentation medium from at least one lignocellulosic material, preferably an industrial and/or agricultural side stream, wherein hemicellulases, in particular selected from xylanase, β-glycosidase, α-arabinofuranosidase, α-glucuronidase, and β-xylosidase, are used at a concentration of between 0.01% and 5% (w/w) and/or at a temperature of between 15 and 100° C. and/or for a time of between 0.5 and 96 hours; In again a further particular embodiment, the present invention relates to a method for the production of a fungal fermentation medium from at least one lignocellulosic material, preferably an industrial and/or agricultural side stream, further comprising the step (a′) of enzymatic hydrolysis of a solid lignocellulosic residue obtained in (a) with cellulase, and separating a liquid product of hydrolysis from a solid residue.

In again a further particular embodiment, the present invention relates to a method for the production of a fungal fermentation medium from at least one lignocellulosic material, preferably an industrial and/or agricultural side stream, wherein (a′) is performed at a temperature of between 15 and 100° C., preferably at a temperature of between 4° and 80° C., and/or at a pH of between 3.0 and 8.0, and/or for a time of between 10 and 200 hours.

In again a further particular embodiment, the present invention relates to a method for the production of a fungal fermentation medium from at least one lignocellulosic material, preferably an industrial and/or agricultural side stream, wherein the industrial and/or agricultural side stream is a solid side stream, wherein preferably the solid side stream is selected from spent grain, cereal brans, cotton, cotton seed husks, bagasse, cocoa shells, cocoa, cocoa pods, cotton and oil press cakes from sunflower, peanut, hazelnut, palm oil, olive, shells and husks from nuts, grass and leaves waste, wood chips, coffee grounds, coffee husks, coffee silverskin, byproducts from the soy industry like soybean pulp (“okara”) and/or rapeseed.

In again a further particular embodiment, the present invention relates to a method for the production of a fungal fermentation medium from at least one lignocellulosic material, preferably an industrial and/or agricultural side stream, further comprising a step of extraction of lipids using supercritical CO₂ and their mechanical separation before the step (a).

In again a further particular embodiment, the present invention relates to a method for the production of a fungal fermentation medium from at least one lignocellulosic material, preferably an industrial and/or agricultural side stream, further comprising a step of removal of toxic compounds present in the aqueous extract of (a) and/or optionally in the liquid product of (a′), such as furfural and/or hydroxymethylfurfural, before step (b).

In again a further particular embodiment, the present invention relates to a method for the production of a fungal fermentation medium from at least one lignocellulosic material, preferably an industrial and/or agricultural side stream, further comprising the step of recovering a solid lignin residue of step (a′).

In again a further particular embodiment, the present invention relates to a method for the production of a fungal fermentation medium from at least one lignocellulosic material, preferably an industrial and/or agricultural side stream, wherein in step (b) the protein composition obtained according to the present invention is further supplemented.

In a further embodiment, the present invention relates to a protein composition obtained according to the method for the production of a fungal fermentation medium from at least one lignocellulosic material, preferably an industrial and/or agricultural side stream of the present invention.

In a further embodiment, the present invention relates to a fungal fermentation medium obtained in the method for the production of a fungal fermentation medium from at least one lignocellulosic material, preferably an industrial and/or agricultural side stream of the present invention.

In a particular embodiment, the present invention relates to the fungal fermentation medium, further supplemented with (a) nitrogen source(s), in particular selected from ammonia, urea, yeast extract, malt extract, corn steep liquor and peptone, and or with a carbon source(s), in particular selected from glucose, fructose, sucrose, lactose, maltose, xylose, galactose, dextrose, glycerol, and molasses, and/or with trace elements and/or vitamins.

In a further particular embodiment, the present invention relates to the fungal fermentation medium, further processed into a dried form.

In a further embodiment, the present invention relates to a method for producing a fungal biomass by submerged fermentation of at least one fungal strain, the method comprising:
(a) providing the pH-adjusted fungal fermentation medium of the present invention to a fermenter suitable for growing fungal mycelium;
(b) cultivating fungal mycelium; and
(c) retrieving and concentrating the fungal biomass to achieve a dry fungal mass content of between 2 to 100%.

In a particular embodiment, the present invention relates to a method for producing a fungal biomass by submerged fermentation of at least one fungal strain, wherein step (b) is performed at a temperature of between 15 and 40° C. and/or at a pH of between 3.0 and 8.5 and/or for a time of between 12 and 240 hours.

In a further particular embodiment, the present invention relates to a method for producing a fungal biomass by submerged fermentation of at least one fungal strain, wherein the at least one fungal strain is an edible fungus.

In again a further particular embodiment, the present invention relates to a method for producing a fungal biomass by submerged fermentation of at least one fungal strain, wherein the at least one fungal strain is selected from Basidiomycota and Ascomycota.

In again a further particular embodiment, the present invention relates to a method for producing a fungal biomass by submerged fermentation of at least one fungal strain, wherein the at least one fungal strain is selected from Pezizomycotina and Agaromycotina In again a further particular embodiment, the present invention relates to a method for producing a fungal biomass by submerged fermentation of at least one fungal strain, wherein the at least one fungal strain is selected from Peziomycetes, Agaricomycetes and Sordariomycetes In again a further particular embodiment, the present invention relates to a method for producing a fungal biomass by submerged fermentation of at least one fungal strain, wherein the at least one fungal strain is selected from Pezizales, Boletales, Cantharellales, Agaricales, Polyporales, Russulales, Auriculariales, Sordoriales and Hypocreales.

In again a further particular embodiment, the present invention relates to a method for producing a fungal biomass by submerged fermentation of at least one fungal strain, wherein the at least one fungal strain is selected from Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, Sordoriaceae, Nectriaceae and Fistulinaceae.

In again a further particular embodiment, the present invention relates to a method for producing a fungal biomass by submerged fermentation of at least one fungal strain, wherein the at least one fungal strain is P. pulmonarius, P. ostreatus, P. citrinopileatus or P. salmoneostramineus.

In again a further particular embodiment, the present invention relates to a method for producing a fungal biomass by submerged fermentation of at least one fungal strain, wherein the at least one fungal strain is M. esculenta, M. angusticeps or M. deliciosa.

In again a further particular embodiment, the present invention relates to a method for producing a fungal biomass by submerged fermentation of at least one fungal strain, wherein the at least one fungal strain is F. venenatum, N. crassa or N. intermedia.

In again a further particular embodiment, the present invention relates to a method for producing a fungal biomass by submerged fermentation of at least one fungal strain, wherein the submerged fermentation is operated as a batch, a fed-batch or a continuous process.

In again a further particular embodiment, the present invention relates to a method for producing a fungal biomass by submerged fermentation of at least one fungal strain, wherein more than one fungal strain are co-fermented.

In a further embodiment, the present invention relates to a fungal biomass produced according to the method for producing a fungal biomass by submerged fermentation of at least one fungal strain of the present invention.

In a particular embodiment, the present invention relates to a fungal biomass produced according to the method for producing a fungal biomass by submerged fermentation of at least one fungal strain of the present invention, wherein the fungal strain is selected from Pleurotaceae, in particular wherein the fungal strain is P. pulmonarius, P. ostreatus, P. citrinopileatus or P. salmoneostramineus.

In a further particular embodiment, the present invention relates to a fungal biomass produced according to the method for producing a fungal biomass by submerged fermentation of at least one fungal strain of the present invention, wherein the fungal strain is selected from Morchellaceae, in particular wherein the fungal strain is M. esculenta, M. angusticeps or M. deliciosa.

In a further embodiment, the present invention relates to use of the fungal biomass of the present invention in production of a fungal-based food product.

In a particular embodiment, the present invention relates to the use of the fungal biomass of the present invention in production of a fungal-based food product, wherein the solid lignin residue recovered according to the present invention is further used in production of the fungal-based food product.

In a further particular embodiment, the present invention relates to the use of the fungal biomass of the present invention in production of a fungal-based food product, wherein the solid lignin residue recovered according to the method for the production of a fungal fermentation medium from at least one industrial and/or agricultural side stream of the present invention is further processed, for example by milling and/or by grinding before being further used in production of the fungal-based food product.

In a further particular embodiment, the present invention relates to the use of the fungal biomass of the present invention in production of a fungal-based food product, wherein the protein composition recovered according to the method for the production of a fungal fermentation medium from at least one industrial and/or agricultural side stream of the present invention is used in the preparation of the fungal based food product.

In a further embodiment, the present invention relates to a fungal-based food product prepared using the fungal biomass of the present invention.

In a particular embodiment, the present invention relates to the fungal-based food product prepared using the fungal biomass of the present invention, wherein the solid lignin residue recovered according to the method for the production of a fungal fermentation medium from at least one industrial and/or agricultural side stream of the present invention is used in the preparation of the fungal-based food product.

In a particular embodiment, the present invention relates to the fungal-based food product prepared using the fungal biomass of the present invention, wherein the protein composition recovered according to the method for the production of a fungal fermentation medium from at least one lignocellulosic material, preferably at least one industrial and/or agricultural side stream of the present invention is used in the preparation of the fungal-based food product.

### Definitions

C5-polysaccharides are defined herein as polysaccharides comprising C5-sugars.

C5 polysaccharides fraction is defined herein as preferably of content of at least 50% of C5-sugars (which may be present as monomers and/or comprised in oligo and/or polysaccharides) as understood as weight/weight ratio of C5-sugars to the total saccharide content, more preferably of content of at least 65% C5 sugars. C5 sugars, or pentoses, are understood herein as sugars having five carbon atoms. It is noted that C5 polysaccharides fraction may contain other sugars, in particular C6 sugars (sugars having 6 carbon atoms, also referred to as hexoses), as monomers and/or comprised within polysaccharides and/or oligosaccharides.

C5-complex polysaccharide fraction relates to C5-polysaccharide fraction as defined herein wherein at least 50%, preferably at least 60%, more preferably at least 70%, even more preferably 80%, even more preferably at least 90% of said C5-sugars are in the form of polysaccharides and/or oligosaccharides.

Polysaccharides are herein understood as molecules comprising more than 1 sugar moieties, connected to each other through glycosidic bond. As used herein, preferably oligosaccharide refers to a polysaccharide having from 2 to 20 sugar moieties. Preferably, polysaccharide has at least 21 sugar moieties.

The food or feed product is defined herein as any product suitable for oral consumption, preferably food, feed, drink or a supplement for food or feed. Thus, the food or feed product should preferably have a taste acceptable to the animal species for which it is intended. Food products for human consumption preferably have a pleasant taste. Pleasant taste may for example be determined by a test panel. As understood by a skilled person, depending on the animal for which the feed or food product is intended it will have a different form.

Functional food as understood herein is defined as any food that goes beyond simple nutrition and has at least one specific targeted action to improve the health and/or well being of the host and/or prevent pathological states in the host.

Mycelium as understood herein refers to a mass (or biomass) of hyphae grown from cells isolated either from the mushroom fruiting body or from the vegetative part of the fungus.

Polysaccharides comprising monosaccharides: A polysaccharide is said to comprise monosaccharides, wherein said monosaccharides are covalently linked to form said polysaccharide. Hydrolysing a polysaccharide will yield the monosaccharides that formed said polysaccharide in free form. The monosaccharide content of a polysaccharide can thus be determined by hydrolysing the polysaccharide and measuring the presence of individual monosaccharides. The monosaccharide content of a mixture of polysaccharides is determined by determining the monosaccharide content of the entire mixture.

The term “polypeptide” as used herein covers proteins, peptides and polypeptides, wherein said proteins, peptides or polypeptides may or may not have been post-translationally modified. Post-translational modification may for example be phosphorylation, methylation, glycosylation, Severity factor also referred to as Ro is herein defined as:

$$Ro = {t}_{R} {e}^{\frac{T - 100}{14.75}}$$

wherein t_{R} is retention time (expressed in minutes), also understood as the time of the process, and T is temperature expressed in ° C.

### BRIEF DESCRIPTION OF FIGURES

FIG. 1: Summary of the contamination experiments performed with the fungal fermentation media of the present invention.

FIG. 2: Comparison of a meatball prepared from mycelium obtained by growing the biomass of P. pulmonarius on a reference medium and on a medium according to the present invention. The recipe for both meatballs is identical; the only difference is the mycelium used in preparation.

FIG. 3: The comparison of the lignocellulosic material after one step thermal extraction and two step thermal extraction. Left is the material after enzymatic hydrolysis when it has only been treated thermally once and right the hydrolyzed material when it has been pretreated twice. We can clearly see that the material that has been pretreated twice is more “powdery” whereas for the other one the fiber structure of the plant material is still very clear. The colour (darker) of the material pretreated twice also shows that we extracted more and “only” lignin (very dark) is remaining.

FIG. 4: The plot summarizing the sensory panel data comparing the meatball obtained from mycelium grown on the medium obtained from the spent grain according to the method of the present invention and from the reference medium.

FIG. 5: The plot showing the relationship between severity of the thermal treatment and the percentage of lysine and aspartate and asparagine in the protein from the obtained extract. The severity can be used to set the concentration of certain amino acids in the extracts and therefore in the medium resulting thereof. This enables the creation of designed extracts for the production of designed fungal biomass.

### DETAILED DESCRIPTION OF THE INVENTION

In one embodiment, the present invention relates to a method for the production of a fungal fermentation medium from at least one lignocellulosic material, preferably at least one industrial and/or agricultural side stream. The method comprises of the steps: (a) aqueous extraction of the at least one lignocellulosic material, preferably at least one industrial and/or agricultural side stream; and (b) combination of the aqueous extract(s) obtained in (a) with optionally at least one nutrient supplement for fungal cultivation.

The at least one lignocellulosic material is preferably herein defined as a material that comprises dry plant matter. Preferably, said lignocellulosic material comprises cellulose, hemicellulose and lignin. Preferably, the at least one lignocellulosic material is at least one industrial and/or agricultural side stream, as defined herein. Further preferably, said lignocellulosic material is preferably solid.

Examples of the lignocellulosic material include spent grain, cereal brans, cotton, cotton seed husks, bagasse, cocoa shells, cocoa, cocoa pods, cotton and oil press cakes from sunflower, peanut, hazelnut, palm oil, olive, shells and husks from nuts, grass and leaves waste, wood chips, coffee grounds, coffee husks, coffee silverskin, rapeseed and byproducts from the soy industry like soybean pulp (“okara”).

The at least one industrial and/or agricultural side stream is not particularly limited and can be any industrial and/or agricultural side stream known to the skilled person. Preferably, the at least one industrial and/or agricultural side stream refers to one industrial and/or agricultural side stream. Preferably the industrial and/or agricultural side stream is a solid side stream. As defined herein, the term solid side stream relates to any side stream that cannot be handled as a liquid, for example cannot be pumped, as opposed to liquid side streams, for example molasse or vinasse, which can be handled as a liquid and, for example, can flow without application of the mechanical forces. The non-limiting examples of solid side stream are given in the following. Further preferably, the solid side stream is selected from spent grain, cereal brans, cotton, cotton seed husks, bagasse, cocoa shells, cocoa, cocoa pods, cotton and oil press cakes from sunflower, peanut, hazelnut, palm oil, olive, shells and husks from nuts, grass and leaves waste, wood chips, coffee grounds, coffee husks, coffee silverskin, rapeseed and/or byproducts from the soy industry like soybean pulp (“okara”). Even more preferably, the solid side stream is spent grain.

The at least one lignocellulosic material, preferably at least one industrial and/or agricultural side stream may also refer to more than one lignocellulosic material, preferably industrial and/or agricultural side stream. For example, it may refer to two, three, four or more lignocellulosic materials, e.g. industrial and/or agricultural side streams. Preferably, the at least one lignocellulosic material, preferably the at least one industrial and/or agricultural side stream comprises spent grain. More preferably, the at least one lignocellulosic material, preferably the at least one industrial and/or agricultural side stream is spent grain.

Herein, spent grain is preferably understood as a leftover or by-product of brewing industry. Preferably, spent grain is a material that remains after the mashing step and has a dry matter content of preferably between 10% and 30%. However, the dry matter content as recited herein is not meant to be limiting, as the skilled person is aware that dry matter content can be increased in preprocessing, for example by pressing, by drying or by other methods that are known to skilled person. Furthermore, the spent grain originating from other industries (for example spent grain obtainable as a byproduct of production of foodstuffs) can also be used within the scope of the present invention.

It should be noted that depending on the industrial and/or agricultural side stream used, extracts and fungal fermentation media with different properties can be obtained.

The present inventors have surprisingly found that when a lignocellulosic material, preferably an industrial and/or agricultural side stream is used as described herein in the preparation of the medium, either the taste, the nutritional profile or the colour of the resulting food product made using the biomass grown on said medium is improved.

In another preferred embodiment, the at least one lignocellulosic material, preferably the at least one industrial and/or agricultural side stream is cocoa shells. Cocoa shells are understood herein as the leftover of cocoa powder production and this material is typically dry (i.e. at least 90% dry mass, or dry biomass, in the material). It comprises the shells encompassing the cocoa beans. Upon application of the methods of the present invention, in particular the method of production of a fungal fermentation medium from at least one lignocellulosic material, preferably at least one industrial and/or agricultural side stream, the cocoa shells may be used in the food production process, despite the technical prejudice that the heavy metal contamination makes the dry powder unsuitable for food application

The lignocellulosic material, preferably the industrial and/or agricultural side stream as used herein can preferably undergo thermal and/or mechanical pretreatment, preferably before being subjected to step (a) as defined above. The aim of thermal and/or mechanical pretreatment step is to increase the efficiency of the subsequent extraction step(s), which are also referred to as (a).

Accordingly, in the mechanical pretreatment the lignocellulosic material, preferably the industrial and/or agricultural side stream is shredded or otherwise broken down into smaller pieces. This step is known to the skilled person and used equipment and exact procedure is selected based on the dry matter of the raw material. For example, bead mills, pin mills or any other kind of mechanical treatment resulting in reduction of particle size of the raw material is useful in the method of the present invention.

Further accordingly, the lignocellulosic material, preferably the industrial and/or agricultural side stream as used herein is contacted with steam, i.e. with water at a temperature of more than 100° C. and at a pressure of more than 1 bar. This step may also be referred to as thermal pretreatment step, or prehydrolysis with steam.

Preferably, the lignocellulosic material, preferably the industrial and/or agricultural side stream as defined herein in accordance with the present invention undergoes prehydrolysis with steam, as defined hereinabove. Preferably, the lignocellulosic material, preferably the industrial and/or agricultural side stream is contacted with steam at the temperature of more than 100° C., preferably at the temperature of between 150° C. and 300° C., more preferably at the temperature of between 160° C. and 180° C., even more preferably at the temperature of about 170° C., for a time of up to 20 minutes, preferably for a time of between 5 and 15 minutes, more preferably for a time of between 7.5 and 15 minutes.

Preferably, the step of prehydrolysis with steam may occur before the step (a) of the method for the production of a fungal fermentation medium of the present invention.

However, preferred are also the embodiments of the method for the production of a fungal fermentation medium according to the present invention wherein the prehydrolysis with steam as defined hereinabove is comprised in step (a) of the method.

Optionally, the lignocellulosic material, preferably industrial and/or agricultural side stream as used herein can undergo pretreatment according to the method selected from washing, solvent-extraction, solvent-swelling, comminution, milling, steam pretreatment, explosive steam pretreatment, dilute acid pretreatment, hot water pretreatment, alkaline pretreatment, lime pretreatment, wet oxidation, wet explosion, ammonia fiber explosion, organosolvent pretreatment, biological pretreatment, ammonia percolation, ultrasound, electroporation, microwave, supercritical CO₂, supercritical H₂O, ozone, and gamma irradiation.

Optionally, before step (a) the water present in the lignocellulosic material, preferably industrial and/or agricultural side stream is removed. This step is preferably performed in the embodiments of the method of the present invention wherein the at least one industrial and/or agricultural side stream is not spent grain. This process is known to the skilled person and can be performed for example by using the screw press (or any other suitable press) to press the side stream. Alternatively, the lignocellulosic material, preferably industrial and/or agricultural sidestream may be dried, for example by using a fluid bed dryer. A convection oven may also be used for this purpose. Preferably in the method of the present invention the so obtained water is discarded and not used further in the method of the present invention. It can however be recovered and used to wash the material subjected previously to thermal prehydrolysis with steam.

Preferably, before the step (a) the lignocellulosic material, preferably industrial and/or agricultural side stream can undergo lipid extraction. Removal of lipids from the lignocellulosic material, preferably from the industrial and/or agricultural side stream can be performed according to any method known to the skilled person. Preferably, lipids may be removed by extraction with supercritical CO₂, as described in DE10201410884. Upon extraction, the lipid fraction is separated from the solid lignocellulosic material, preferably industrial and/or agricultural side stream, which is further processed. The so obtained lipid fraction can be used in other methods of the present invention.

According to the method for the production of a fungal fermentation medium from at least one lignocellulosic material, preferably at least one industrial and/or agricultural side stream, the method comprises the step (a) of aqueous extraction of the at least one lignocellulosic material, preferably industrial and/or agricultural side stream. Herein, aqueous extraction is preferably understood as extraction with liquid water. In the method of the present invention, temperature and pressure are preferably so selected that water is in the liquid state, despite in certain cases the temperature exceeding 100° C. Skilled person is capable of determining if this position is fulfilled based on the phase diagram of water.

As understood herein, the step (a) of aqueous extraction of the at least one lignocellulosic material, preferably at least one industrial and/or agricultural side stream is performed in a suitable reactor, known to the skilled person. The at least one lignocellulosic material, preferably industrial and/or agricultural side stream is loaded into the said reactor at the solid load preferably between 5 and 70% weight/volume (w/v), preferably between 10 and 55% (w/v) and treated with water, preferably with liquid water. The solid load as understood herein is defined as the ratio of weight of dry solid side stream (the material loaded) to the complete reaction volume (including the water used for extraction and the at least one solid side stream, and preferably expressed as percentage. The weight of material loaded is herein understood as the dry weight. The solid load as required in the present invention depends on the material loaded and the reactor characteristics. As understood to the skilled person, the amount of material loaded preferably should not affect the stirring and the heat transfer inside the reactor. It also depends on the amount of liquid extract that has to be recovered as well as its composition. As understood herein, the reactor can preferably be loaded up to 55% w/v with dry lignocellulosic biomass (also referred to as dry solid side stream). It is noted that in certain embodiments, alternatively, the reactor can be preferably loaded with wet lignocellulosic biomass. As known to the skilled person, the preferred load depends on the material, i.e. lignocellulosic material, preferably industrial and/or agricultural side stream. For example, preferred load for spent grain is from 5% to 35%, more preferably about from 10 to 20%, whereas preferred load for cocoa shells is from 40% to 50%. As further known to the skilled person, the preferred load depends on the type of the reactor, and the material loaded (i.e. type of side stream and its processing). For example, reactors with mechanical stirring may be able to handle higher loads than those with magnetic stirring.

If prehydrolysis with steam is performed, the reactor needs to be loaded only with the lignocellulosic material (e.g. an industrial and/or agricultural side stream). Preferably, the reactor is loaded so that the preferred solid load as defined hereinabove is calculated for the washing step following the prehydrolysis with steam. It is to be understood herein that the prehydrolysis with steam and the following washing step (washing with liquid water) preferably take place in the same reactor.

Preferably, the water used in the aqueous extraction step as described herein is at a pressure of between 2 to 220 bar, more preferably of between 10 and 220 bar. Even more preferably, the water used in the aqueous extraction step as described herein is at a pressure of between 10 and 50 bar. Even more preferably, the water used in the aqueous extraction step as described herein is at a pressure of between 40 and 50 bar.

Preferably, prehydrolysis with steam as defined herein is performed at the pressure of between 1 and 20 bar, more preferably 1 and 10 bar. If the lignocellulosic material is spent grain, the pressure of steam for prehydrolysis with steam is preferably between 4 and 15 bar.

Preferably, the water used in the aqueous extraction step as described herein is at a temperature of between 9° and 374° C. More preferably, the water used in the aqueous extraction step as described herein is at a temperature of between 10° and 350° C. Even more preferably the water used in the aqueous extraction step as described herein is at a temperature of between 10° and 250° C.

In certain embodiments, wherein extraction is performed at a temperature of more than 180° C., furfural that is formed under these conditions needs to be removed and is removed according to the methods known to the skilled person.

In a preferred embodiment, the water used in the aqueous extraction step as described herein is at a temperature of between 140° C. and 180° C., and/or at a pressure of between 20 and 50 bar, preferably of between 35 and 50 bar, more preferably of between 40 and 50 bar. Preferably, the extraction is performed for a time of between 10 and 60 minutes. It is noted that these conditions are particularly suitable for an embodiment wherein the at least one lignocellulosic material, preferably industrial and/or agricultural side stream is spent grain. It is further noted that under these conditions no production of furfural is observed.

In another preferred embodiment, the water used in the aqueous extraction step as described herein is at a temperature of between 10° and 140° C. and/or at a pressure of between 2 and 25 bar. Preferably, the extraction is performed for a time of between 10 and 90 minutes. It is noted that these conditions are particularly suitable for an embodiment wherein the at least one industrial and/or agricultural side stream is spent cocoa shells. It is further noted that under these conditions no production of furfural is observed.

In the method of the present invention, the aqueous extraction of the at least one lignocellulosic material, preferably industrial and/or agricultural side stream is preferably performed for a time of between 10 and 200 minutes. More preferably, the aqueous extraction of the at least one lignocellulosic material, preferably industrial and/or agricultural side stream is performed for a time of between 10 and 120 min. Most preferably, the aqueous extraction of the at least one lignocellulosic material, preferably industrial and/or agricultural side stream is performed for a time of between 10 and 60 minutes. Preferably, the water as used for the extraction is to be maintained in liquid form.

Particularly preferred is an extraction process that combines prehydrolysis with steam with extraction/washing step performed with liquid water. Preferably, the lignocellulosic material, preferably the industrial and/or agricultural side stream, is contacted with steam at the temperature of more than 100° C., preferably at the temperature of between 150° C. and 300° C., more preferably at the temperature of between 160° C. and 180° C., even more preferably at the temperature of about 170° C., for a time of up to 20 minutes, preferably for a time of between 5 and 15 minutes, more preferably for a time of between 7.5 and 15 minutes. Afterwards, the so treated solid is washed with liquid water, preferably at the temperature of 50° C. to 100° C., more preferably at the temperature of 50° C. to 70° C., even more preferably at the temperature of 50° C. to 60° C. In said process, preferably a single extract is produced in step (a). Preferably, said extract is a result of washing with liquid water, as described hereinabove. Preferably, the washing step as defined herein is performed for the time of between 5 and 60 minutes.

Preferably, the step (a) as described herein comprises only prehydrolysis with steam as described hereinabove, and washing with liquid water, as described hereinabove.

In one embodiment of the present invention, the water used for prehydrolysis with steam may comprise diluted acid, for example not more than 1% w/w of said acid. Particularly suitable are preparations of H₂SO₄ at 0.2, or 0.4% w/w.

In certain embodiments of the present invention, step (a) may comprise prehydrolysis with steam as described hereinabove, washing with liquid water, as described hereinabove, and the second step of prehydrolysis with steam, which is preferably to be performed when further processing of the lignocellulosic residue is to be performed (see also FIG. 3 for the effects of the second prehydrolysis step).

According to the present invention, the conditions of the extraction, in particular the temperature, the pressure, and the time of the extraction is set so that preferably at least 10%, at least 20%, at least 30%, at least 40%, at least 50%, at least 60%, at least 70% or at least 80% of saccharides contained in the lignocellulosic material, preferably in the industrial and/or agricultural side stream is recovered in the aqueous extraction. Preferably, the time of the extraction as well as number of extraction steps are set so that preferably at least 40%, more preferably at least 60% of C5-polysaccharides contained in the lignocellulosic material, preferably industrial and/or agricultural side stream is recovered in the aqueous extraction. As understood by the skilled person, the time of the extraction may depend on further conditions, in particular on the applied reactor (in particular in the context of available stirring, as discussed herein), as well as on the particular type of material (the at least one lignocellulosic material, preferably industrial and/or agricultural side stream).

Alternatively, the conditions of the extraction, in particular the temperature, the pressure, and the time of the extraction is set so that preferably at least 10%, at least 20%, at least 30%, at least 40% or at least 50% of proteins contained in the lignocellulosic material, preferably industrial and/or agricultural side stream is recovered in the aqueous extraction. Preferably, the time of the extraction as well as number of extraction steps are set so that preferably at least 10%, preferably at least 20%, more preferably at least 30% of proteins contained in the lignocellulosic material, preferably industrial and/or agricultural side stream is recovered in the aqueous extraction.

Preferably, step (a) of aqueous extraction of a lignocellulosic material, preferably industrial and/or agricultural side stream according to the present invention is performed with water at a pH of between 2.0 and 12.0, preferably 3.0 and 10.0, more preferably 4.0 and 8.0, even more preferably 5.0 and 8.0. The pH values as understood herein are measured under a pressure of 1.0 bar and temperature of 25° C., even though the extraction itself is performed under different conditions, as disclosed herein. Preferably, the pH is adjusted before the water is placed in contact with the at least one lignocellulosic material, preferably industrial and/or agricultural side stream. It is further understood herein, that addition of acid or base to water as described herein to a final concentration of more than 1% is preferably to be avoided.

In the method for the production of a fungal fermentation medium from at least one lignocellulosic material, preferably at least one industrial and/or agricultural side stream, the step (a) of the aqueous extraction of the at least one lignocellulosic material, preferably industrial and/or agricultural side stream can also comprise more than one extraction steps. In such a situation, more than one extract is produced. Preferably, the step (a) can comprise two extraction steps, referred to herein as (a1) and (a2). Preferably step (a1) is performed first, and step (a2) is performed afterwards.

Preferably, step (a1) comprises extraction of the lignocellulosic material, preferably industrial and/or agricultural side stream with water at a temperature of between 90 to 374° C., more preferably of between 10° and 220° C., even more preferably of between 110 to 180° C. Preferably, step (a1) comprises extraction of the lignocellulosic material, preferably industrial and/or agricultural side stream with water at a pressure of 2 and 220 bar, more preferably at a pressure of between 6 and 35 bar, even more preferably, a pressure of between 20 and 35 bar. Step (a1) is preferably performed for a time of between 10 to 200 minutes, more preferably for a time of between 10 and 60 minutes. Preferably, the water herein is supplemented by NaOH at a concentration of between 0.1% to 1.0% w/w.

The extract obtained in the step (a1), in particular wherein the water is supplemented by NaOH, preferably contains proteins extracted from the lignocellulosic material, preferably industrial and/or agricultural side stream. The extract obtained in the step (a1) may also be referred to as protein-rich extract. Preferably, the content of protein in the extract obtained in the step (a1) is at least 5 g/L. The said extract is separated from the lignocellulosic material, preferably solid industrial and/or agricultural side stream (which is to be understood as the remains of said material after extraction) by any suitable separation technique known to the skilled person. Preferably, a decanter centrifuge is used for this purpose. The solid residue, i.e. the lignocellulosic material, preferably the industrial and/or agricultural side stream that has undergone aqueous extraction according to step (a1) is further produced in this step. Such a solid product may also be referred to as a solid lignocellulosic residue.

Preferably, said extract obtained in the step (a1) comprises C5-sugars as (complex) C5-polysaccharides as defined herein. Preferably, at least 50% of C5 sugars is in a form of polysaccharides, more preferably at least 60% of C5 sugars is in a form of polysaccharides, even more preferably at least 70% of C5 sugars is in a form of polysaccharides, even more preferably at least 80% of C5 sugars is in a form of polysaccharides, even more preferably at least 90% of C5 sugars are in the form of polysaccharides.

The aqueous extract obtained in the step (a1) can be further processed. Optionally, the aqueous extract obtained in step (a1) is separated from the solid residue, by the techniques known to the skilled person. Optionally, proteins present in the aqueous extract obtained in (a1), preferably upon separation, can be isolated by using the techniques known to the skilled person. Preferably, the proteins present in the aqueous extract obtained in (a1) can be isolated by flocculation or by precipitation with CO₂.

Obtained product comprising proteins is separated from the liquid by using the suitable separation techniques known to the skilled person and can be further applied in the method of the present invention, as disclosed herein. Obtained product comprising proteins may be also referred to as a protein composition, obtainable according to the method of the present invention. As understood herein, the obtained product can also comprise sugars and other components, its composition is not meant to be limited to proteins. It is noted that liquid remaining after separation of proteins as describe herein can be further used in step (b) as an extract obtained in step (a).

Alternatively, the method for the production of a fungal fermentation medium from at least one lignocellulosic material, preferably at least one industrial and/or agricultural side stream of the present invention may further comprise a step wherein proteins present in the aqueous extract obtained in (a1) are hydrolyzed before the step (b). To this end, the extract obtained in (a1) is further treated with proteolytic enzymes (proteases, peptidases) to yield an extract comprising amino acids and/or peptides. The time of hydrolysis depends on the desired composition of the product.

In certain embodiments of the present invention, the obtained solid protein product or extract comprising proteins and/or amino acids and/or peptides can be concentrated, in particular through evaporation, or it can be dried (for example it can be freeze-dried) in order to remove excess liquid and make the product suitable for long-term storage.

The solid residue, i.e. the lignocellulosic material, preferably the industrial and/or agricultural side stream, preferably spent grain, that has undergone aqueous extraction according to step (a1), is preferably subjected to step (a2), as defined herein. Preferably, step (a2) comprises extraction of the lignocellulosic material, preferably the industrial and/or agricultural side stream with water at a temperature of between 12° and 220° C., more preferably of between 13° and 200° C. Preferably, step (a2) comprises extraction of the lignocellulosic material, preferably the industrial and/or agricultural side stream with water at a pressure of between 1.25 bar and 220 bar, preferably at a pressure of between 2 and 220 bar, more preferably at a pressure of between 6 and 35 bar, even more preferably, a pressure of between 20 and 35 bar. Step (a1) is preferably performed for a time of between 5 and 200 minutes, preferably for a time of between 10 and 200 minutes, more preferably for a time of between 10 and 100 minutes.

As understood herein, step (a2) is preferably performed to prepare the cellulose structure for efficient hydrolysis.

The extract obtained in the step (a2), contains C5-polysaccharides extracted from the lignocellulosic material, preferably the industrial and/or agricultural side stream. The extract obtained in the step (a2) may also be referred to as C5-complex polysaccharide fraction. Preferably, said fraction comprises C5-sugars as (complex) C5-polysaccharides as defined herein. Preferably, at least 50% of C5 sugars is in a form of polysaccharides, more preferably at least 60% of C5 sugars is in a form of polysaccharides, even more preferably at least 70% of C5 sugars is in a form of polysaccharides, even more preferably at least 80% of C5 sugars is in a form of polysaccharides, even more preferably at least 90% of C5 sugars are in the form of polysaccharides. The said extract is separated from the solid lignocellulosic material, preferably the industrial and/or agricultural side stream (preferably from its remains after the extraction process) by any suitable separation technique known to the skilled person. Preferably, a decanter centrifuge is used for this purpose. As it is understood, said remains after the extraction process typically consist mostly of lignin.

The method for the production of a fungal fermentation medium from at least one lignocellulosic material, preferably at least one industrial and/or agricultural side stream, may optionally further comprise a step wherein C5-polysaccharides present in the aqueous extract obtained in (a2) are further hydrolyzed to monosaccharides optionally using hemicellulases before the step (b). As disclosed herein, the hemicellulases, in particular selected from xylanase, β-glycosidase, α-arabinofuranosidase, α-glucuronidase, and β-xylosidase, are used at a concentration of between 0.01% and 5% (w/w) and/or at a temperature of between 15 and 100° C. and/or for a time of between 0.5 and 96 hours.

As it is understood by the skilled person, the so prepared extract would no longer comprise C5-polysaccharides and C5-sugars would preferably be present in a hydrolyzed form (preferably mostly as monosaccharides).

As disclosed herein, the step (a) of the method for the production of a fungal fermentation medium from at least one lignocellulosic material, preferably at least one industrial and/or agricultural side stream of the present invention, may involve the steps of:
(a1) extraction of the lignocellulosic material, preferably industrial and/or agricultural side stream with water, optionally supplemented by NaOH at a concentration of between 0.1% to 1.0% w/w, at a temperature of between 90 to 374° C., preferably of between 10° and 220° C., more preferably of between 100 to 180° C., for a time of between 5 and 200 minutes, preferably for a time of between 10 to 200 minutes; and
(a2) extraction of the lignocellulosic material, preferably industrial and/or agricultural side stream with water at a temperature of between 12° and 220° C., preferably of between 13° and 200° C., for a time of between 5 and 150 minutes.

As known to the skilled person, extraction conditions may also be referred to and described by the severity factors. Accordingly, preferably the step (a) of the method for the production of a fungal fermentation medium from at least one lignocellulosic material, preferably industrial and/or agricultural side stream of the present invention, may involve the steps of:
(a1) extraction of the lignocellulosic material, preferably the industrial and/or agricultural side stream with water, optionally supplemented by NaOH at a concentration of between 0.1% to 1.0% w/w, preferably wherein the temperature and time of the extraction correspond to a severity factor of between 0.7 and 10.4, preferably of between 1.0 and 5.8, more preferably of between 1.0 and 4.7, and
(a2) extraction of the lignocellulosic material, preferably industrial and/or agricultural side stream with water, preferably wherein temperature and time of the extraction correspond to a severity factor of between 1.3 and 5.7, preferably of between 1.6 and 5.1.

By performing step (a) of the method of the present invention as two steps (a1) and (a2), as defined herein, the recovery of C5-polysaccharides is improved as compared to a single step extraction. Preferably, at least 15% of C5-polysaccharides as defined herein are recovered. Furthermore, the production of furfural, which is undesired, is reduced.

The present inventors have demonstrated that by performing the step (a) as two steps (a1) and (a2) as disclosed herein the efficiency of optional enzymatic hydrolysis steps afterwards is improved. FIG. 3 shows the comparison of the material after a single and double heat pretreatment steps, accordingly.

In certain embodiments, the step (a) of aqueous extraction of the at least one lignocellulosic material, preferably at least one industrial and/or agricultural side stream may be divided in several steps. Each of the steps constituting step (a) of the method of the present invention may be performed as described herein. Increasing number of steps constituting step (a), as known to the person skilled in the art of extraction, may lead to increased yield of extraction, which is herein preferably understood as higher total amount of recovered polysaccharides and/or recovered proteins in the process. An increase recovery of polysaccharides may also lead to a cleaner and purer lignin product after the enzymatic treatment of step (a′). Furthermore, when one extraction step (a) is divided into several steps, production of toxic materials including furfural and/or hydroxymethylfurfural is significantly reduced.

Undesired presence of furfural and/or hydroxymethylfurfural and/or other undesired compounds may be avoided, as described above. Furthermore, undesired furfural and/or hydroxymethylfurfural and/or other undesired compounds may be removed, for example by gas stripping, by heteroazeotropic distillation or by liquid-liquid extraction. Optionally, furfural may be recovered and used for other industrial applications.

The present invention further relates to a method for the production of a fungal fermentation medium from at least one lignocellulosic material, preferably industrial and/or agricultural side stream, further comprising steps of processing the aqueous extract(s) obtained in (a) before the step (b).

The processing the aqueous extract(s) obtained in (a) before the step (b), which as understood herein is an optional step, may preferably include separation of proteins from the aqueous extracts of (a). Any separation method suitable for the purpose and known to the skilled person can be used in the method of the present invention.

Preferably, the said proteins are separated from the aqueous extract(s) preferably by flocculation or by precipitation with CO₂, preferably followed by mechanical separation, for example with a decanter centrifuge. Optionally, the so obtained proteins may be further prepared as a solution comprising not less than 50% w/w of polypeptides and/or amino acids. Further optionally, the so obtained proteins are hydrolyzed, preferably by using proteolytic enzymes, in particular selected from alcalases, papain, proteinase K, and trypsin. The proteolytic enzymes can be used, at a concentration of between 0.01% and 5% (w/w) (which is understood herein as a total concentration of all the enzymes used herein) and/or at a temperature of between 15 and 100° C. and/or for a time of between 0.5 and 96 hours. The so obtained solution(s) containing hydrolyzed protein(s) (i.e. polypeptide(s) and amino acids) can optionally be further used in step (b) of the method of the present invention.

Preferably, in the method for producing the fungal fermentation medium according to the present invention, the proteins are not separated from the extract, i.e. from the product of the said method. The present inventors have found it to be beneficial to keep the proteins, as defined herein, in the medium as nitrogen source. As understood herein, the proteins may comprise amino acid, peptides and/or proteins.

Upon separation of proteins, the so obtained solution contains C5-polysaccharides. The C5-polysaccharides (preferably in complex form) present in the solution upon protein separation can be optionally hydrolyzed, at least in part, to monosaccharides. Preferably, hemicellulases are used for this purpose. Preferably, as disclosed herein, the hemicellulases, in particular selected from xylanase, β-glycosidase, α-arabinofuranosidase, α-glucuronidase, and β-xylosidase, are used at a concentration of between 0.01% and 5% (w/w) and/or at a temperature of between 15 and 100° C. and/or for a time of between 0.5 and 96 hours. The monosaccharides obtained herein include xylose and/or arabinose. The so obtained solution(s) can optionally be further used in step (b) of the method of the present invention. Also encompassed within the scope of the present invention is hydrolysis using chemical agent, for example by using acid, for example HCl, at a concentration of preferably up to 0.1 M, or by using base, for example NaOH, at a concentration of preferably up to 0.1 M.

In addition to the extract(s) obtained in step (a) as disclosed herein, a solid lignocellulosic residue remains as a product of extraction. According to the present invention, the solid lignocellulosic residue obtained in (a) may be further used in the method of the present invention or may be disposed of. Thus, preferably, the method for the production of a fungal fermentation medium from at least one lignocellulosic material, preferably industrial and/or agricultural side stream of the present invention may further comprise the step (a′) of enzymatic hydrolysis of a solid lignocellulosic residue obtained in (a) with cellulase, and separating a liquid product of hydrolysis from a solid residue. Accordingly, the lignocellulosic residue is loaded into the second reactor (preferably at a load of between 5 and 50% w/w) where it is hydrolysed with cellulase. Preferably, the step (a′) is performed at a temperature of between 15 and 100° C., more preferably at a temperature of between 4° and 80° C. Preferably, the step (a′) is performed at a pH between 3.0 and 8.0, more preferably at a pH between 4.0 and 7.0. Preferably, the step (a′) is performed for a time of between 10 and 200 hours.

The method for the production of a fungal fermentation medium from at least one lignocellulosic material, preferably from at least one industrial and/or agricultural side stream of the present invention may optionally further comprise the step of recovering a solid lignin residue of step (a′). Solid lignin residue (or solid lignocellulosic residue), as defined herein, may be separated from the extracts by using the methods known to the skilled person, for example by using a decanter centrifuge.

As known to the skilled person, certain compounds formed during the extraction process, in particular during the extraction process at elevated temperatures, are detrimental to the performance of a fungal fermentation medium. Such compounds include furfural and/or hydroxymethylfurfural, and may collectively be referred to as toxic compounds. As disclosed herein, by performing step (a) as more than one step, for example as steps (a1) and (a2), as disclosed herein, the temperature can be controlled and kept lower, so that formation of toxic compounds like furfural and/or hydroxymethyl furfural can be minimized or avoided. Alternatively, the method for the production of a fungal fermentation medium from at least one lignocellulosic material, preferably at least one industrial and/or agricultural side stream of the present invention may preferably further comprise a step of removal of toxic compounds present in the aqueous extract of (a) and/or optionally in the liquid product of (a′), before step (b) of the method of the present invention. The methods for removal of toxic furfural are known to the skilled person.

It is further noted that the use of prehydrolysis with steam also reduces the production of toxic compounds, like furfural, as they are volatile compounds and are removed with the steam.

Aqueous extraction step(s) (a) may be performed by any technical method, as known to the skilled person. For example, the aqueous extraction step (or each of the step(s)) can be performed as a batch process. Preferably however, aqueous extraction step(s) (a) of the method of the present invention are to be performed as a continuous extraction process, as known to the skilled person. In a preferred embodiment, liquid water as defined herein is used to extract a fraction comprising C5 polysaccharides (preferably C5-complex polysaccharides fraction) and proteins, whereto enzymes as defined herein are optionally added to hydrolyse polysaccharides at least in part to monosaccharides.

As understood herein, aqueous extracts obtainable in the step (a) of the method of the present invention comprise C5-polysaccharides (preferably in complex form) and proteins.

The method for the production of a fungal fermentation medium from at least one lignocellulosic material, preferably at least one industrial and/or agricultural side stream of the present invention optionally further comprises a step (b) wherein the aqueous extract(s) of step (a) obtained according to the present invention is(are) further supplemented. The aqueous extract(s) of step (a) obtained according to the present invention can be further supplemented with nitrogen source(s), carbon source(s), trace element(s), vitamin(s) and/or protein composition(s). The nitrogen sources as defined herein are preferably selected from ammonia, urea, yeast extract, malt extract, corn steep liquor and peptone. More preferably, the nitrogen source(s) are ammonia and/or urea. The carbon source(s) are preferably selected from glucose, fructose, sucrose, lactose, maltose, xylose, galactose, dextrose, glycerol, and molasses, more preferably the carbon source is glucose. The trace element(s) as defined herein may include for example iron(Ill) salts, copper(II) salts, zinc salts, manganese(II) salts, molybdenum salts and/or cobalt(II) salts. Vitamins as defined herein preferably include vitamins that are beneficial for the growth of fungi on the medium obtainable according to the method of the present invention, for example folic acid, riboflavin, pantothenic acid or biotin. Protein composition may be further used to supplement the aqueous extract of (a) of the present invention. Preferably, protein composition obtainable from proteins separated from the aqueous extract(s) of (a) of the method of the present invention, preferably by flocculation or by precipitation with CO₂, are preferably used in the method of the present invention.

Preferably, according to step (b) no further carbon source is added to the extract of step (a). Further preferably, according to step (b) the extract of step (a) is preferably supplemented with at least one nitrogen source, as described hereinabove.

Preferably, the extract(s) obtained in the step (a) constitute(s) at least 50% of the final product of step (b), more preferably at least 70%, even more preferably at least 90%. It is to be understood that preferably the % values refer to w/w % of the remaining solid after water removal for extract(s) of step (a) and the final product of step (b).

As understood herein, the product of the step (b) of the method of the present invention may be further processed. Optionally, the water contained in the product of step (b) may be removed, for example by spray-drying, drum-drying, belt-drying, or freeze-drying, yielding dried fungal fermentation medium, which as known to the skilled person may have improved shelf time. Optionally, the fungal fermentation medium of the present invention may be further sterilized or pasteurized within the scope of the method of the present invention. Optionally, the fungal fermentation medium obtainable according to the method of the present invention may be further supplemented, for example by salts (preferably sodium chloride, sodium nitrate, magnesium sulfate, calcium chloride, calcium carbonate, ammonium chloride, diammonium phosphate, ammonium sulfate, potassium phosphate, disodium phosphate, and/or monosodium phosphate), antibiotics, or by water. The water, as used herein, is preferably used to optimize the concentration of the components in the medium.

Further preferably, the pH of the fungal fermentation medium obtainable in the method for the production of a fungal fermentation medium from at least one lignocellulosic material, preferably industrial and/or agricultural side stream of the present invention can be set to a desired value preferably by addition of buffering agents. Particularly useful herein are citrate or phosphate buffer systems. Further, in the fermenter the pH can be adjusted by addition of the appropriate amount of urea, NaOH, ammonia, sulfuric acid, phosphoric acid, or hydrochloric acid.

It should however be mentioned, that in certain embodiments the fungal fermentation medium obtained after step(s) (a) of the method for the production of a fungal fermentation medium from at least one lignocellulosic material, preferably at least one industrial and/or agricultural side stream of the present invention is suitable for supporting fungal fermentation and could be used without further steps, preferably also without step (b).

Thus, the fungal fermentation medium from at least one lignocellulosic material, preferably at least one industrial and/or agricultural side stream of the present invention is preferably used upon addition of solid nitrogen source(s), as defined in step (b)

In a further embodiment, the present invention relates to a fungal fermentation medium obtainable in the method for the production of a fungal fermentation medium from at least one lignocellulosic material, preferably at least one industrial and/or agricultural side stream of the present invention. The fungal fermentation medium of the present invention obtainable in the method for the production of a fungal fermentation medium from at least one lignocellulosic material, preferably industrial and/or agricultural side stream of the present invention may optionally further comprise nitrogen source(s), carbon source(s), trace element(s), vitamin(s) and/or protein composition(s). The nitrogen sources as defined herein are preferably selected from ammonia, urea, yeast extract, malt extract, corn steep liquor and peptone. More preferably, the nitrogen source(s) are ammonia and/or urea. The carbon source(s) are preferably selected from glucose, fructose, sucrose, lactose, maltose, xylose, galactose, dextrose, glycerol, and molasses, more preferably the carbon source is glucose or xylose. The trace element(s) as defined herein may include for example iron(Ill) salts, copper(II) salts, zinc salts, manganese(II) salts, molybdenum salts and/or cobalt(II) salts. Vitamins as defined herein preferably include vitamins that are beneficial for the growth of fungi on the medium obtainable according to the method of the present invention, as defined herein.

Preferably, the fungal fermentation medium of the present invention comprises C5-polysaccharides. Preferably, the C5 polysaccharides constitute at least 50% w/w of all the sugars in said medium, more preferably the C5-polysaccharides constitute at least 65% w/w of all the sugars in said medium, even more preferably the C5-polysaccharides constitute at least 80% w/w of all the sugars in said medium. This preferably applies to the medium prepared according to the present invention, wherein the optional step (a′) has not been performed.

As understood herein, C5-polysaccharides are preferably a major form of C5-sugars in the medium of the present invention. C5-polysaccharides preferably constitute more than 50% of all C5-sugars in said medium. As understood herein, presence of the C5-sugars in C5-polysaccharide form makes them suitable for fungal fermentation.

In certain embodiments, the C5-polysaccharides are hydrolyzed to monosaccharides. Preferably, according to the optional step (a′). In such a case, the fungal fermentation medium comprises C5-sugars. Preferably, the C5-sugars constitute at least 10% w/w of all the sugars in said medium, more preferably the C5-sugars comprise at least 20% w/w of all the sugars in said medium, even more preferably the C5-sugars comprise at least 30% w/w of all the sugars in said medium.

The fungal fermentation medium of the present invention may be optionally further processed as defined herein. In particular, within the scope of the present invention the fungal fermentation medium as described herein may be further processed into a dried form. To this end, the water contained in the fungal fermentation medium obtainable according to the present invention may be removed, for example by spray-drying, belt-drying, drum-drying or freeze-drying, yielding dried fungal fermentation medium, which as known to the skilled person may have improved shelf-life. Optionally, the fungal fermentation medium of the present invention may be further sterilized within the scope of the method of the present invention.

The fungal fermentation medium, as disclosed herein, is usable in fungal fermentation. Accordingly, the fungal fermentation medium as disclosed herein is usable in the method for producing a fungal biomass of the present invention. Therefore, in a further embodiment, the present invention relates to a method for producing a fungal biomass by submerged fermentation of at least one fungal strain.

The said method for producing a fungal biomass by submerged fermentation of at least one fungal strain comprises the following steps:
(a) providing the pH-adjusted fungal fermentation medium obtainable according to the method of the present invention to a fermenter suitable for growing fungal mycelium;
(b) cultivating fungal mycelium; and
(c) retrieving and concentrating the fungal biomass to achieve a dry fungal biomass content of between 2 to 100%.

Dry fungal biomass is also understood as biomass of fungi upon drying, upon removal of excess water that can be removed without breaking the cells. Dry fungal biomass content in the fungal biomass is defined as ratio between the weight of dry fungal biomass as defined herein, and the fungal biomass before drying, as obtained in step (c).

As understood herein, submerged fermentation or submerged fungal fermentation is defined as cultivation of fungi in the liquid medium. As known to the skilled person, an alternative to submerged fungal fermentation is surface fungal fermentation, also referred to as solid state fungal fermentation. The liquid fungal fermentation medium, herein as understood the fungal fermentation medium of the present invention that has been pH adjusted (see below) in solution or suspension is placed in an enclosed vessel, herein preferably a fermenter, which is usually sterilized to kill organisms that may interfere with fungal growth, according to the methods known to the skilled person. An inoculum of the at least one fungal strain as defined herein is introduced into the vessel (herein preferably fermenter) and, at least in the case of aerobic fungi, air is blown into the vessel. The contents of the vessel (fermenter herein) are preferably stirred according to the methods known to the skilled person, and preferably that can be integrated in the fermenter design. Stirring brings nutrients present in the medium and oxygen in continuous contact with the matter being fermented (herein the at least one fungal strain) and, preferably, temperature and pH are controlled at levels suitable to the fungus. After certain time, typically after between 1 to 12 days, depending on the type of fermentation, fungus, and exact fermentation conditions, among others, the fungal biomass can be harvested. (as noted by the skilled person, the timing as given herein may not necessarily apply to the cases of continuous fermentation). As however known to the skilled person, mixing may also be achieved by other methods than stirring, which may also influence the morphology of the fungal cells, as well as lead to subjecting the fungal cells to the shear stress. As understood herein, method of mixing is not meant to be limiting, and any applicable method known to the skilled person falls within the scope of the present invention.

In the first step of the method for producing a fungal biomass by submerged fermentation of at least one fungal strain, the pH-adjusted fungal fermentation medium obtainable according to the method of the present invention is provided to a fermenter suitable for growing fungal mycelium. Suitable fermenters are known to the skilled person. For example, a suitable stirred tank with a specific stirrer useful in reducing the shear stress, or an airlift fermenter, is useful within the scope of the present invention. The fungal fermentation medium is understood as the medium obtainable according to the methods of the present invention and disclosed herein.

As understood herein, the fungal fermentation medium can be further sterilized in certain embodiments of the present invention. As known to the skilled person, sterilization may be done by exposing the medium to elevated temperature for certain period of time. Typically, it is performed at a temperature of between 15° and 200° C. for a time of between 30 s and 10 minutes. However, the conditions as recited herein are not meant to be limiting for the scope of the present invention.

In the next step of the method, the step (b), the fungal mycelium is cultivated. Preferably, step (b) is performed at a temperature of between 15 and 40° C. Typically, a constant temperature is maintained throughout the process, which as known to the skilled person may be selected for optimal growth of a particular fungal strain. For example, in the case of P. ostreatus the step (b) is preferably performed at a temperature of between 25 and 30° C. Further preferably, step (b) is performed at a pH of between 3.0 and 8.5. The pH of the medium can be adjusted within the scope of the method for production of the fungal fermentation medium of the present invention, and/or within the scope of step (a) of the method of producing a fungal biomass of the present invention. As understood to the skilled person, selection of pH may be dependent on the fungal strain to be cultivated, or on potential contaminating strains to be excluded from growing. Further preferably, the step (b) is performed for a time of between 12 and 240 hours. As however understood to the skilled person, if step (b) is performed as a continuous process, then it preferably will not be limited to 240 hours.

As understood herein, selection of the growth conditions, for example including pH, fungal fermentation medium and/or temperature, may affect the growth of the fungal mycelium, metabolism of the fungal cells, and/or whether the fungus grows as pellet or as a mycelium.

Within the scope of the method for producing a fungal biomass by submerged fermentation of at least one fungal strain of the present invention, it is understood that the at least one fungal strain is an edible fungus. Edible fungus is herein understood as a fungus that can be consumed by a mammal as food, preferably by a human, without causing any adverse reaction. Adverse reactions are herein defined as food poisoning, or undesirable taste properties that would preclude consumption. Edible fungus is herein not limited to its fruiting bodies (mushrooms), but other parts of the fungus, for example mycelium, can also be considered as an edible mushroom.

In the method for producing a fungal biomass by submerged fermentation of at least one fungal strain of the present invention, the at least one fungal strain is selected from Basidiomycota and Ascomycota.

According to the present invention, the at least one fungal strain can be selected from Basidiomycota. Preferably, the at least one fungal strain can be selected from Basidiomycota can be a fungal strain selected from Agaromycotina. As defined herein, a fungal strain selected from Agaromycotina can be a fungal strain selected from Agaricomycetes. Preferably, a fungal strain selected from Agaricomycetes can be a fungal strain selected from Boletales, Cantharellales, Agaricales, Polyporales, Russulales, and Auriculariales.

As defined herein, the fungal strain selected from Boletaceae is preferably B. edulis.

In certain embodiments, a fungal strain selected from Agaricomycetes can be a fungal strain selected from Polyporales. Preferably, as defined herein, a fungal strain selected from Polyporales can be a fungal strain selected from Meripilaceae, Polyporaceae, Ganodermataceae, Sparassidaceae As defined herein, a fungal strain selected from Meripilaceae is preferably G. frondosa.

As defined herein a fungal strain selected from Polyporaceae is preferably selected from P. umbellatus and L. sulphureus. As defined herein a fungal strain selected from Sparassidaceae is preferably S. crispa.

Preferably, a fungal strain selected from Agaricomycetes can be a fungal strain selected from Cantharellales. Further preferably, a fungal strain selected from Cantharellales can be a strain selected from Cantharellaceae and Hydnaceae. As defined herein, a strain selected from Cantharellaceae can be C. cornucopioides or C. cibarius, preferably C. cibarius. As further defined herein, a strain selected from Hydnaceae can be H. repandum.

Alternatively, a fungal strain selected from Agaricomycetes can be a fungal strain selected from Boletales. Preferably, as defined herein, a fungal strain selected from Boletales can be a fungal strain selected from Boletaceae, and Sclerodermataceae.

Alternatively, a fungal strain selected from Agaricomycetes can be a fungal strain selected from Russulales. Further preferably, as defined herein, a fungal strain selected from Russulales can be a fungal strain selected from Hericiaceae, and Bondarzewiaceae. Preferably, a fungal strain selected from Russulales is a fungal strain selected from Hericiaceae, preferably selected from H. erinaceus and H. coralloides. Further preferably, the fungal strain selected from Bondarzewiaceae is B. berkeleyi.

Alternatively, a fungal strain selected from Agaricomycetes can be a fungal strain selected from Auriculariales, more preferably a fungal strain selected from Auriculariaceae. Preferably, a fungal strain selected from Auriculariaceae is A. auricula-judae.

Preferably, in the method of the present invention the at least one fungal strain is selected from Agaricales. Accordingly, the at least one fungal strain selected from Agaricales can be selected from Tuberaceae, Pleurotaceae, Agaricaceae, Marasmiaceae, Strophariaceae, Lyophyllaceae, Tricholomataceae, Omphalotaceae, Physalacriaceae, Schizophyllaceae, and Fistulinaceae.

As defined herein, the fungal strain selected Marasmiaceae from is preferably L. edodes. As further defined herein, the fungal strain selected from Strophariaceae is preferably a fungal strain selected from A. aegerita and H. capnoides. As further defined herein, the fungal strain selected from Lyophyllaceae is preferably C. Indica.

As further defined herein, the fungal strain selected from Tricholomataceae is preferably a fungal strain selected from H. tesselatus and C. nuda. As further defined herein, the fungal strain selected from Omphalotaceae is preferably C. gigantean. As further defined herein, the fungal strain selected from Physalacriaceae is preferably F. velutipes. As further defined herein, the fungal strain selected from Schizophyllaceae is preferably S. commune. As further defined herein, the fungal strain selected from Fistulinaceae is preferably F. hepatica.

The at least one fungal strain according to the present invention selected from Agaricales can be selected from Tuberaceae. Preferably, the fungal strain according to the present invention selected from Tuberaceae is T. magnatum, T. estivum, T. uncinatum, T. indicum, T. rufum or T. melanosporum, more preferably T. melanosporum and T. magnatum.

More preferably, the at least one fungal strain selected from Agaricales can be a fungal strain selected from Pleurotaceae. Even more preferably, the at least one fungal strain of the present invention is a fungal strain selected from P. pulmonarius, P. ostreatus, P. citrinopileatus and P. salmoneostramineus, even more preferably selected from P. pulmonarius or P. ostreatus, most preferably P. pulmonarius.

The at least one fungal strain according to the present invention selected from Agaricales can be selected from Agaricaceae. Preferably, the fungal strain selected from Agaricaceae as defined herein is A. bisporus or A. blazei, more preferably A. bisporus.

According to the present invention, the at least one fungal strain can be selected from Ascomycota. Preferably, the at least one fungal strain can be selected from Ascomycota can be a fungal strain selected from Pezizomycotina. As defined herein, a fungal strain selected from Pezizomycotina can be selected from Pezizomycetes.

Preferably, within the scope of the present invention, a fungal strain selected from Pezizomycetes can be a fungal strain selected from Pezizales.

Further preferably, the at least one fungal strain as defined in the method for production of fungal biomass of the present invention can be selected from Pezizales. Preferably, the fungal strain selected from Pezizales can be selected from Morchellaceae.

Preferably, the fungal strain selected from Morchellaceae is M. esculenta, M. angusticeps or M. deliciosa.

Alternatively, the at least one fungal strain selected from Ascomycota can be a fungal strain selected from Sordariomycetes. Preferably, at least one strain as defined herein, selected from Sordariomycetes, can be a fungal strain selected from Hypocreales.

Further preferably, a fungal strain selected from Hypocreales can be a fungal strain selected from Cordycipitaceae. Even further preferably, a fungal strain selected from Cordycipitaceae is a fungal strain selected from C. militaris and C. sinensis.

Alternatively, a fungal strain selected from Hypocreales can be a fungal strain preferably selected from Nectriaceae. Further preferably, the fungal strain selected from Nectriaceae can be a Fusarium strain. In another embodiment, the fungal strain selected from Sordariomycetes can be a fungal strain selected from Sordariaceae.

Further preferably, the fungal strain selected from Sordariaceae can be a Neurospora strain.

As disclosed herein, in the method for producing a fungal biomass by submerged fermentation of at least one fungal strain of the present invention, the at least one fungal strain can be selected from Pezizomycotina and Agaromycotina.

As further disclosed herein, in the method of the present invention for producing a fungal biomass by submerged fermentation of at least one fungal strain, the at least one fungal strain is preferably selected from Peziomycetes, Agaricomycetes and Sordariomycetes.

As further disclosed herein, in the method of the present invention for producing a fungal biomass by submerged fermentation of at least one fungal strain, the at least one fungal strain is preferably selected from Pezizales, Boletales, Cantharellales, Agaricales, Polyporales, Russulales, Auriculariales, Sordoriales and Hypocreales.

As further disclosed herein, in the method of the present invention for producing a fungal biomass by submerged fermentation of at least one fungal strain, the at least one fungal strain is preferably selected from Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, and Fistulinaceae.

As further disclosed herein, in the method of the present invention for producing a fungal biomass by submerged fermentation of at least one fungal strain, the at least one fungal strain is preferably P. pulmonarius, P. ostreatus, P. citrinopileatus or P. salmoneostramineus, even more preferably P. pulmonarius or P. ostreatus.

As further disclosed herein, in the method of the present invention for producing a fungal biomass by submerged fermentation of at least one fungal strain, the at least one fungal strain is preferably M. esculenta, M. angusticeps or M. deliciosa.

The method for producing a fungal biomass by submerged fermentation of at least one fungal strain of the present invention is not limited to the fermentation of a single fungal strain. It is also encompassed within the present invention that more than one fungal strains are co-fermented, as described herein. Selection of strains for co-fermentation depends on their compatibility and can be performed by a skilled person.

In the method for producing a fungal biomass by submerged fermentation of at least one fungal strain of the present invention the submerged fermentation can be operated as a batch, a fed-batch or a continuous process. These three main methods of fermentation are known to the skilled person and differ by outflow and inflow of material from/to the fermentation vessel.

The batch processes are characterized by lack of inflow of material into the fermentation vessel. In a batch process, all nutrients are provided at the beginning of the cultivation, without adding any more in the subsequent bioprocess. During the entire bioprocess, no additional nutrients are added with the exception of gases, acids and bases. The bioprocess then lasts until the nutrients are consumed. This strategy is suitable for rapid experiments such as strain characterization or the optimization of nutrient medium. The disadvantage of this convenient method is that the biomass and product yields are limited. Since the carbon source and/or oxygen transfer are usually the limiting factor, the microorganisms are not in the exponential growth phase for a long time. After the end of a bioprocess run in batch mode, only the biomass or medium is harvested and appropriately processed to obtain the desired product. From the bioreactor point of view, the process is repeatedly interrupted by cleaning and sterilization steps, and the biomass is only produced in stages.

In the fed batch process, substrate, nutrients and other substances may be added into the fermentation vessel, to extend the possible culture time or increase the yield, among others. The advantage of feeding during cultivation is that it allows to achieve higher product quantities overall. Under specific growth conditions, the microorganisms and/or cells constantly double and therefore follow an exponential growth curve. Therefore, in certain embodiments the feed rate may be increased exponentially as well. Generally, the substrate is pumped from the supply bottle into the culture vessel, for example through a silicone tube. The user can either manually set the feed at any time (linear, exponential, pulse-wise), or add nutrients when specific conditions are met, such as when a certain biomass concentration is reached or when a nutrient is depleted. The fed-batch process offers a wide range of control strategies and is also suitable for highly specialized applications. However, it may increase the processing time and potentially leads to inhibition through the accumulation of toxic by-products.

Preferably, in the method of the present invention the submerged fermentation is operated as a continuous process. After a batch growth phase, an equilibrium is established with respect to a particular component (also called steady state). Under these conditions, as much fresh culture medium is added, as it is removed (chemostat). These bioprocesses are referred to as continuous cultures, and are particularly suitable when an excess of nutrients would result in inhibition due to e.g. acid or ethanol build up or excessive heating. Other advantages of this method include reduced product inhibition and an improved space-time yield. When medium is removed, cells are harvested, which is why the inflow and outflow rates must be less than the doubling time of the microorganisms. Alternatively, the cells can be retained in a wide variety of ways (for example, in a spin filter), which is called perfusion. In a continuous process, the space-time yield of the bioreactor can be even further improved compared to that of a fed-batch process. However, the long cultivation period also increases the risk of contamination and long-term changes in the cultures. In the method of the present invention, as discussed herein, as the extract obtained in step (a) of the method of the present invention preferably comprises more C5-polysaccharides than C6-polysaccharides by weight, the medium preferably supports the growth of fungi, over for example bacteria, and is therefore particularly suitable for continuous fermentation method. This is preferably the case wherein step (a′) of the method of the present invention is not performed. Furthermore, in the method of the present invention, as discussed herein, as the extract obtained in step (a) of the method of the present invention preferably comprises C5-polysaccharides, the medium preferably supports the growth of fungi, over for example bacteria, and is therefore particularly suitable for continuous fermentation method. Furthermore, as understood herein, providing fungal fermentation medium comprising C6 polysaccharides will promote growth of fungal strains able to produce cellulase, that are able to grow on C6-polysaccharides as a carbon source. The three most common types of continuous culture are chemostat (The rate of addition of a single growth-limiting substrate controls cell multiplication), turbidostat (an indirect measurement of cell numbers −turbidity or optical density—which needs an additional sensor but is driven by real-time feedback, controls addition and removal of liquid), and perfusion (this type of continuous bioprocessing mode is based on either retaining the cells in the bioreactor or recycling the cells back to the bioreactor; fresh medium is provided and cell-free supernatant gets removed at the same rate).

In a further embodiment, the present invention relates to a fungal biomass produced according to the method for producing a fungal biomass by submerged fermentation of at least one fungal strain of the present invention. Preferably, the fungal biomass comprises the fungal cells of the fungal strain selected from Pleurotaceae, in particular wherein the fungal strain is P. pulmonarius, P. ostreatus, P. citrinopileatus or P. salmoneostramineus, more preferably selected P. pulmonarius or P. ostreatus. In another embodiment, preferably the fungal biomass comprises the fungal cells of the fungal strain selected from Morchellaceae, wherein the fungal strain is M. esculenta, M. angusticeps or M. deliciosa. However, the fungal biomass of the present invention is not limited to a single fungal strain. It is also encompassed within the present invention that more than one fungal strain are co-fermented to yield the fungal biomass of the present invention, as described herein. Selection of strains for co-fermentation depends on their compatibility and can be performed by a skilled person. Furthermore, selection of strains for co-inclusion in the fungal biomass of the present invention depends on their properties and envisaged application, as well as their growth rates, as disclosed herein.

The fungal biomass of the present invention preferably has a protein content between 10 and 60% (w/w). As further disclosed herein, the fungal biomass of the present invention preferably has a fiber content between 20 and 60% (w/w).

In a further embodiment, the present invention relates to use of the fungal biomass of the present invention in production of a fungal-based food product. Accordingly, the present invention also relates to a fungal-based food product, obtainable as described herein. The fungal-based food product of the present invention may be prepared in any form known to the skilled person. For example, the fungal-based food product of the present invention may take the form of a ball (i.e. meatball replacement), dumpling, vegetarian sausage, meat-replacement steak, meat-replacement ground meat product, meat-replacement product for preparing sandwiches, etc.

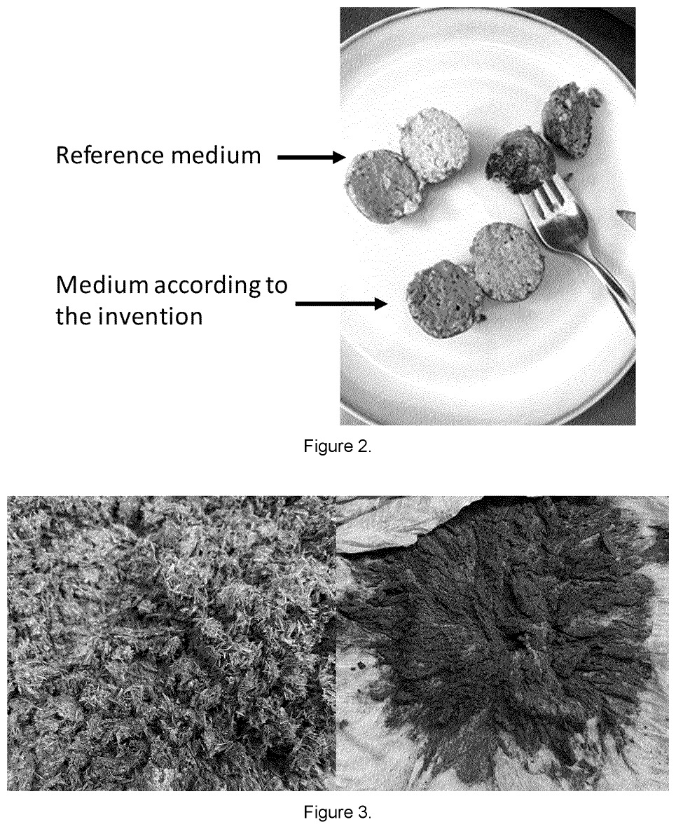

The present inventors have shown that the biomass of the present invention, grown on the medium of the present invention is darker and hence closely resembles the minced meat that the biomass obtainable from the growth on the reference medium (see FIG. 2). The present inventors have shown that this is particularly the case in an exemplary case wherein the medium of the present invention is obtainable in the method for the production of a fungal fermentation medium of the present invention wherein the step (a) as described herein comprises only prehydrolysis with steam as described hereinabove, and washing with liquid water, as described hereinabove. The present inventors have further shown that this is the case wherein the lignocellulosic material, e.g. an industrial and/or agricultural side stream, is spent grain. The present inventors have thus shown that the taste, appearance and/or nutritional profile of the obtained food product is affected by introducing the prehydrolysis of a lignocellulosic material with steam step into the method for preparing said medium, and further may depend on a specific lignocellulosic material used.

Similar effects have also been shown extracting cocoa shell with water to obtain extracts and a biomass composition that are different than the ones obtained on the reference medium or extract from spent grain (see Example 4 and Tables 10, 11 and 12 therein)

The food product according to the present invention may for example be a nutritional supplement. The nutritional supplement could be in the form of a liquid or a solid, such as a pill, lozenge or tablet. For example, the nutritional supplement of the present invention may be a protein supplement and/or a carbohydrate supplement.

The food product as understood herein may be a dairy product, for example yoghurt, milk drinks and ice cream. The food product as understood herein may also relate to different embodiments of seafood products, for example a crabcake, fishcake, tuna, salmon, or shrimp.

The food product may be texturized food product or a textured food product. Accordingly, the food product of the present invention comprises all amino acids necessary for human daily intake that cannot be synthetized in novo. Furthermore, the textured food product of the present invention is preferably heat-resistant, boiling resistant and suitable for cooking. For example, the fungal-based food product of the present invention, as described herein, may be a meat replacement product. It is noted that preferably the meat replacement product is a texturized food product or textured food product. It is further noted that the structure of the textured food product improves the acceptability of the textured food product by consumers. It is further noted that intrinsic fibrous texture of the fungal biomass of the present invention maybe beneficial for producing a textured food product or a texturized food product without using conventional texturizing methods such as extrusion.

In a particular embodiment, the fungal-based food product produced using the fungal biomass obtainable according to the methods of the present invention, is further supplemented with the solid lignin residue obtained according the methods for production of the fungal fermentation medium of the present invention. In other words, the solid lignin residue obtained according the methods for production of the fungal fermentation medium of the present invention is further used in production of the fungal-based food product. the solid lignin residue obtained according the methods for production of the fungal fermentation medium of the present invention can be used as a food additive, as texturizer or as an aroma carrier in the production of the fungal-based food product of the present invention. In particular, the present invention encompasses the inclusion of the solid lignin residue to a content of between 0 and 30% w/w of dry solid lignin residue in a fungal based food-product of the present invention. As understood herein, the solid lignin residue obtained according to the method for the production of a fungal fermentation medium from at least one lignocellulosic material, preferably industrial and/or agricultural side stream of the present invention can be further processed, for example by milling and/or by grinding, or by using other methods known to the skilled person, before being further used in production of the fungal-based food product. Accordingly, the lignin may also be further functionalized.

The fungal-based food product of the present invention may be further processed and or supplemented. For example, the fungal based food product of the present invention may be further supplemented with water, salt, oil and/or spices, according to protocols known to the skilled person. Further processing may also include heat and high-pressure treatment (in particular useful for a high-pressure pasteurization), brewing, boiling, baking, frying, fermenting, and/or drying of the food product. As known to the skilled person, preservatives may be added to lengthen the shelf life of the food product of the present invention.

According to the present invention, the protein composition obtained according to the method for the production of a fungal fermentation medium from at least one lignocellulosic material, preferably industrial and/or agricultural side stream of the present invention can be further used in the preparation of the fungal based food product. In other words, the fungal based food product, prepared using the fungal biomass of the present invention is further supplemented with the protein composition obtained according to the method for the production of a fungal fermentation medium from at least one lignocellulosic material, preferably industrial and/or agricultural side stream of the present invention.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples are merely illustrative of the present invention and should not be construed to limit the scope of the invention which is defined by the appended claims in any way.

### EXAMPLES

### General Experimental Protocols

### Thermal Extraction of the Lignocellulosic Material

For thermal extraction, the dry matter of the lignocellulosic material was determined with a moisture analyser (160° C. until constant weight) using 3 g of the material. Based on the obtained dry matter, the reactor load was adjusted by weighing the required amount of material and introducing it to the reactor afterwards. Water was then added to fill up the reactor to its final working volume and a rubber sealing ring was placed on top of the reactor housing. The lid which is equipped with a manometer and temperature probe was then tightly closed to avoid leakages and the reactor was then pressurized with nitrogen until the desired operation pressure is reached. After that, the desired temperature, pressure, and retention time were set in the software and extraction was started. During the experiment, temperature and pressure were constantly monitored and regulated.

At the end of the extraction, the reactor was cooled down by submerging it in cool water or, for larger reactors, using the cooling jacket. Once the temperature decreased below 30° C., the reactor was depressurized by opening slowly the exhaust valve and the lid opened as soon as the pressure reaches atmospheric pressure. In the case of a continuous extraction process, the mixture was cooled after extraction from the reactor using a heat exchanger. The solid and liquid phase (extract) were then separated either by press filtration or with a decanter centrifuge. The liquid extracts were then either shortly stored at 4° C. or freeze at −20° C. for long time storage. The remaining treated solid lignocellulosic material was further submitted to enzymatic hydrolysis to recover glucose from cellulose.

### Hydrolysis of the Lignocellulosic Material

The dry matter of the recovered lignocellulosic material after treatment as described in the section “Thermal extraction of the lignocellulosic material” was determined using a moisture analyser (incubation at 160° C. until constant weight) and the load was adjusted as described herein for the thermal treatment. The material was then loaded into the reaction vessel and the enzyme cocktail (Ctec2 and Ctec3 from Novozymes) added according to the cellulose content in the material and following the manufacturer's guidelines. The mixture of enzyme and cellulosic material was then incubated for a time between 10 and 200 hours at a temperature of 60° C. and a pH of 6.0. The homogeneity of the mixture was ensured either by a stirrer or by incubating the vessel in an incubator. After the incubation, the reaction mixture was quickly cooled using either an ice bath, the cooling jacket of the reaction vessel or in case of a continuous process, a heat exchanger. Finally, the liquid and solid fractions were separated by press filtration or centrifugation. The solid fraction rich in lignin was stored at 4° C. before analysis and the cleared liquid fraction used for fermentation experiments.

### Analysis of Sugars and Furfural Content

For C5 extracts, i.e. as obtainable according to the section “Thermal extraction of the lignocellulosic material”, the samples were hydrolysed with hydrochloric acid (final concentration 4% w/w) at 121° C. for 60 min before analysis to be able to quantify the recovered C5 sugars as monosaccharides. After hydrolysis, the pH of the extract was then set to 5.5 to avoid damaging the HPLC column. The monosaccharides from C6 extracts, as obtainable according to the section “Hydrolysis of the lignocellulosic material and from C5 extracts as described above, were then measured with an Agilent HPLC 1200 system using a Metacarb 87C column (300×7.8 mm, Varian Inc, Paolo Alto, CA, USA) as the stationary and ultrapure water as the mobile phase. The measurement was performed at a temperature of 85° C. and an isocratic flow of 0.6 mL min⁻¹. After column separation, the analytes were detected by a refractive index detector, except in the case of furfural that was measured using a UV-detector at a wavelength of 270 nm.

### Analysis of Amino Acid Content

The amino acid profile of proteins extracted from the lignocellulosic material was determined by hydrolysing samples of extracts at 105° C. for 24 h with 6 M HCl prior to HPLC measurement. Hydrolysates were subsequently evaporated under a nitrogen stream and resuspended in 200 μM α-aminobutyrate, the latter serving as internal standard. Amino acid concentrations in the prepared samples were finally measured by fluorescence detection using an Agilent 1200 HPLC system (Agilent technologies, Waldbronn, Germany) equipped with a reverse phase column Gemini 5μ C18 110 A (150×4.6 mm, Phenomenex, Aschaffenburg, Germany) as stationary phase. Separation of the different proteinogenic amino acids relied on a gradual change of the mobile phase composition throughout the measurement, mixing differently eluent A (40 mM NaH2PO4, pH 7.8) and eluent B (45% methanol, 45% acetonitrile, 10% water) according to a well-defined gradient profile. Moreover, column separation was operated at 40° C. with a flow rate of 1 mL min⁻¹. In addition, a pre-column (Gemini C18, MAX, RP, 4×3 mm, Phenomenex, Aschaffenburg, Germany) was used to increase column lifetime. Fluorescence detection was achieved through pre-column derivatisation with o-phtalaldehyde (OPA) and 9-fluorenylmethyloxycarbonyl (FMOC) and modification of the excitation and emission wavelength (Table 1).

TABLE 1
Method used for separation and quantification of amino
acids - Composition of the mobile phase was varied
during measurement to achieved separation by gradient elution. Eluent
A: 40 mM NaH₂PO₄, PH 7.8; Eluent B: 45% methanol,
45% acetonitrile, 10% water.

| Time | Eluent A | Eluent B | Excitation λ | Emission λ |
|---|---|---|---|---|
| [min] | [%] | [%] | [nm] | [nm] |
| 0 | 100 | 0.0 | 340 | 450 |
| 40.5 | 59.5 | 40.5 | 340 | 450 |
| 41 | 39 | 61 | 340 | 450 |
| 43 | 39 | 61 | 266 | 308 |
| 57.5 | 0.0 | 100 | 266 | 305 |
| 59.5 | 0.0 | 100 | 340 | 450 |
| 80.5 | 25.0 | 75.0 | 340 | 450 |
| 61.5 | 50.0 | 50.0 | 340 | 450 |
| 62.5 | 75.0 | 25.0 | 340 | 450 |
| 63.5 | 100.0 | 0.0 | 340 | 450 |
| 65.5 | 100.0 | 0.0 | 340 | 450 |

### Thermal Pretreatment and Extraction of Lignocellulosic Biomass Using Steam

Prior to operation, the reactor is preheated with steam until the operational temperature is reached. During the preheating phase the condensation, resulting from the steam getting in contact with the cold reactor, must be removed.

Material is weighted and loaded in the reactor either in a metal cartridges (batch treatment) or using a screw feeder (continuous treatment). The material is pretreated, for the desired residence time with constant injection of steam, the temperature inside the reactor is controlled by setting the pressure controller to the steam pressure corresponding to the desired temperature. In the case of the continuous treatment, the residence time is controlled with the rotation speed of the screw conveyor reactor.

After the residence time is achieved, the material is recovered out of the reactor, its weight and moisture content are recorded to further continue with the next treatment process (extraction).

Extraction is performed in a separate unit, steam-pretreated material is mixed with warm-water (around 50-60° C.) in the desired ratio in order to achieve a certain solid load. Constant mixing is provided by a stirred tank for around 20 minutes before the liquid and solid fractions are separated.

The solid-liquid mixture is pumped to a pressing machine where the two fractions are separated at a constant pressure of around 4 bar. The liquid hydrolysate (rich is C5 sugars) as well as the solid fraction (rich in cellulose and lignin) are recovered for further treatment.

The solid fraction can then be further treated with enzymes for the conversion of cellulose into C6 sugars.

### Cultivation of Fungal Biomass

The liquid extracts obtained after thermal extraction were either used directly as a growth medium, mixed and/or supplemented with additional compounds (5.9 g/I K₂HPO₄, 9.0 g/L KH₂PO₄, 12.0·10⁻² g/L MgSO₄, 8.10-10·3 FeCl₃, 10.10·10⁻³ CaCl₂) and other trace elements according to typical M9 medium (Miller, 1972, Experiments in Molecular Genetics. Cold Spring Harbor, NY: New York Cold Spring Harbor Laboratory). The obtained mixture was then placed in appropriate vessel and sterilised (121° C., 20 min). After sterilisation, the medium was let to cool to room temperature and inoculated with spores or mycelium from a suspension prepared from a fully grown mycelium agar plate. The broth was then incubated at a temperature of 30° C. for a time of 5 days. The pH was regulated using acid and base during fermentation to keep it at the optimal pH of 6.5 for the cultivated strain of P. pulmonarius. After that, the biomass was harvested by centrifugation, washed with water and the dry matter finally determined using a moisture analyser (160° C. until constant weight). The dry matter was converted to a biomass concentration using the broth volume.

The reference biomass used for comparison with biomass produced from extracts was produced using a reference medium consisting of glucose, a trace solution containing magnesium, iron, manganese, zinc, copper and calcium as well as yeast extract as nitrogen source. The pH of the medium was adjusted to 6.5 using phosphate buffer and flasks were incubated at 30° C. for 5 days.

Sensory Evaluation of Meatballs Produced with Mycelium from Different Origins

Meatballs were formed from mycelium biomass and fried in a pan. Each trained panellist was blindfolded and successively received a meatball prepared with mycelium either from the standard medium and one from spent grain. During this first session, they defined the sensory attributes they recognised in the two meatballs. Subsequently, they discussed the attributes together and chose common attributes that every panellist can associate to the same taste and aroma of the meatballs to compare them. A second session was then started, and the panellists had now to evaluate the meatballs according to the chosen attributes and put a score between 0 and 5 for each attribute. This session was repeated on different days to increase statistical relevance of data and average of scoring were calculated and plotted on a spider web.

The colour was determined using the RGB system and a colour analyser at 20 different positions on the meatballs. The positions used for the 10 measurements were the same for all meatballs. The mean values of these measurements were then used to compare the colour of the meatballs.

### Example 1—Production of Fungal Cultivation Medium from Spent Grain

Extraction experiments were performed on spent grain using ten different conditions as defined in Table 2 below, according to the general protocol as outlined in the section “Thermal extraction of the lignocellulosic material” and biomass was subsequently produced using the protocol “Cultivation of fungal biomass”.

TABLE 2
Summary of the extraction conditions for spent grain

| | Treatment Parameters | | |
|---|---|---|---|
| Experiment | Temperature | Time | Severity |
| [—] | [° C.] | [min] | log (Ro) |
| 1 | 180 | 20 | 3.65652549 |
| 2 | 150 | 50 | 3.171 |
| 3 | 150 | 90 | 3.426 |
| 4 | 170 | 10 | 3.061 |
| 5 | 200 | 10 | 3.944 |
| 6 | 120 | 30 | 2.066 |
| 7 | 140 | 20 | 2.479 |
| 8 | 150 | 20 | 2.773 |

The recovery of C5-polysaccharide fraction and protein fraction, as well as the yield of biomass production per solid sidestream loaded (standardization for comparison purposes) from these experiments are summarized in Table 3. Methods for the determination of sugar content are described in “Analysis of sugar and furfural content”.

TABLE 3
Summary of the extraction experiments on spent grain

| | Furfural | C5 sugars - | Protein - | Biomass on C5 |
|---|---|---|---|---|
| | [g/L per % | Recovery | Recovery | [g/L per % |
| Condition | load] | [%] | [%] | load] |
| 1 | 0.070 | 65.38 | 44.96 | n.d. |
| 2 | 0.000 | 39.83 | 24.06 | 1.78 |
| 3 | 0.000 | 30.51 | 17.74 | 1.55 |
| 4 | 0.000 | 30.90 | 25.91 | 1.74 |
| 5 | 0.087 | 32.51 | 57.10 | n.d. |
| 6 | 0.000 | 5.37 | 6.38 | 0.46 |
| 7 | 0.000 | 18.81 | 11.38 | 0.64 |
| 8 | 0.000 | 25.87 | 14.66 | 0.77 |

The extracts have been subjected to the analysis of the amino acid content according to the general procedure as described herein in the section “Analysis of the amino acid content”. The results are summarized in Table 4. It is noted that obtained extracts are characterized by high methionine content as well as by high tyrosine content. It also appears that the amino acid composition depends on the extraction conditions.

Especially, lysine content tends to increase with decreasing severity of the thermal treatment while Aspartate content tends to follow the opposite trend (FIG. 5). Therefore, applying different treatment will enable the creation of different fermentation media and as a consequence the production of different protein profile for the biomass obtained afterwards.

TABLE 4
Summary of amino acid content analysis (protein in the extract)

| Condition | Severity | Asp/Asn | Gln/Glt | Ser | His | Gly | Thr | Arg | Ala |
|---|---|---|---|---|---|---|---|---|---|
| 1 | 3.65652549 | 63.82% | 1.28% | 3.66% | 0.76% | 11.63% | 1.52% | 3.13% | 6.20% |
| 2 | 3.171 | 38.95% | 1.85% | 3.44% | 1.56% | 10.24% | 6.00% | 3.51% | 5.38% |
| 3 | 3.426 | 43.97% | 1.41% | 3.69% | 1.27% | 10.44% | 1.55% | 2.34% | 5.57% |
| 4 | 3.061 | 39.37% | 1.83% | 3.22% | 1.63% | 10.73% | 1.48% | 2.34% | 6.23% |
| 5 | 3.944 | 39.90% | 2.10% | 3.41% | 0.89% | 13.57% | 1.42% | 2.66% | 5.53% |
| 6 | 2.066 | 7.12% | 21.97% | 3.27% | 4.12% | 8.51% | 1.18% | 4.88% | 21.91% |
| 7 | 2.479 | 22.65% | 7.19% | 4.01% | 1.49% | 10.33% | 0.00% | 5.70% | 13.44% |
| 8 | 2.773 | 34.99% | 6.13% | 3.93% | 3.21% | 10.66% | 1.31% | 4.21% | 14.69% |

| Condition | Severity | Tyr | Val | Met | Phe | Ile | leu | Lys |
|---|---|---|---|---|---|---|---|---|
| 1 | 3.656525491 | 1.26% | 2.72% | 0.00% | 1.25% | 0.00% | 1.98% | 0.81% |
| 2 | 3.171 | 2.43% | 3.24% | 8.26% | 2.50% | 5.83% | 3.74% | 3.07% |
| 3 | 3.426 | 11.32% | 2.87% | 7.89% | 1.67% | 1.51% | 2.90% | 1.62% |
| 4 | 3.061 | 12.32% | 3.59% | 8.00% | 1.74% | 2.39% | 2.76% | 2.38% |
| 5 | 3.944 | 11.25% | 3.30% | 11.40% | 1.18% | 1.38% | 2.00% | 0.00% |
| 6 | 2.066 | 3.23% | 2.88% | 0.00% | 4.62% | 0.00% | 2.26% | 14.05% |
| 7 | 2.479 | 1.51% | 4.90% | 8.13% | 9.31% | 1.60% | 3.10% | 6.64% |
| 8 | 2.773 | 2.59% | 2.83% | 0.00% | 2.87% | 0.00% | 2.62% | 9.96% |

### Example 2—Hydrolysis of Cellulose from Pretreated Spent Grain from Example 1

The solid lignocellulosic materials recovered after thermal extraction of spent grain of Example 1, herein the spent grain, was treated according to the methods as described in the section “Hydrolysis of lignocellulosic material”. Recovery of C6-polysaccharides fraction determined as described in “Analysis of sugar and furfural content” as well as the biomass obtained (expressed as per % solid loaded for comparison purposes) from fermentation trials with these extracts performed as described in the protocol “cultivation of fungal biomass” are summarized in Table 5.

TABLE 5
Summary of hydrolysis experiments performed
on the pretreated spent grain from example 1

| | C6 sugar - | C6 sugar recovery | Biomass on C6 |
|---|---|---|---|
| Condition | Recovery [%] | [g/L per % load] | [g/L per % load] |
| 1 | 100 | 3.86475 | n.d |
| 2 | 72.6 | 2.45175 | 1.17 |
| 3 | 89.94171891 | 3.123142857 | 1.74 |
| 4 | 96.5 | 3.018571429 | 2.05 |
| 5 | 86.7 | 3.738285714 | n.c |
| 6 | 91.51375086 | 2.315555556 | 1.50 |
| 7 | 67.44284851 | 2.081 | 0.68 |
| 8 | 75.11933099 | 2.431 | 1.41 |

### Example 3—Two Step Extraction of Spent Grain

Extraction experiments were performed on spent grain using four different two-step protocols as defined in Table 6. After step 1, four different steps 2 were performed—Step 2-1, Step 2-2, Step 2-3 and Step 2-4. Extraction experiments were performed according to the general protocol as outlined in the section “Thermal extraction of the lignocellulosic material”. As it can be seen in Table 6, the two-step protocol wherein the second step (Step 2) was performed at higher temperature has allowed for a better recovery of proteins (compare Step 2-1 and Step 2-2). It is noted that obtained extracts are characterized by high methionine content. It is also noted that the second extraction enables to increase the protein recovery by up to 78.5% and the C5 recovery by almost 100%, thus providing in most cases a suitable medium for fermentation as shown by the biomass recovery (expressed as per % solid sidestream loaded for comparison purposes) listed in Table 6.

TABLE 6
Summary of protein recovery during the extraction steps

| | | | C5 sugar - | Protein - | Biomass on C5 |
|---|---|---|---|---|---|
| | Temperature | Time | Recovery | Recovery | [g/L per % |
| Condition | [° C.] | [min] | [%] | [%] | load] |
| Step 1 | 150 | 50 | 32.907 | 22.06 | 1.073 |
| Step 2 - 1 | 150 | 50 | 8.876 | 17.313 | 0.420 |
| Step 2 - 2 | 130 | 30 | 23.381 | 9.494 | 0.688 |
| Step 2 - 3 | 110 | 90 | 8.660 | n.d. | 0.323 |
| Step 2 - 4 | 170 | 10 | 32.474 | n.d. | 0.870 |

The extracts obtained after the first and second thermal pretreatment (only Step 2-1 and Step 2-2) were submitted for amino acid analysis and results are shown in Table 7. As observed for the experiments with one step extraction, we can see that the amino acid profile of protein is changing depending on the extraction conditions and can therefore be fine-tuned by adjusting the parameters. This feature is particularly interesting for adjusting medium composition for the growth of certain fungi and to alter the composition of the biomass obtained after fermentation. In particular, we observed the same relationship between severity and concentration as for example 1 for aspartate/asparagine and lysine.

TABLE 7
Summary of amino acid content analysis

| Condition | Asp/Asn | Gln/Glt | Ser | His | Gly | Thr | Arg | Ala | Tyr |
|---|---|---|---|---|---|---|---|---|---|
| Step 1 | 38.95% | 1.85% | 3.44% | 1.56% | 10.24% | 6.00% | 3.51% | 5.38% | 2.43% |
| Step 2 - 1 | 37.5% | 2.1% | 3.9% | 1.8% | 11.6% | 6.5% | 4.0% | 5.1% | 2.1% |
| Step 2 - 2 | 51.9% | 1.9% | 3.8% | 1.2% | 11.1% | 4.8% | 2.0% | 4.5% | 1.6% |

| Condition | Val | Met | Phe | Ile | leu | Lys |
|---|---|---|---|---|---|---|
| Step 1 | 3.24% | 8.26% | 2.50% | 5.83% | 3.74% | 3.07% |
| Step 2 - 1 | 3.0% | 8.2% | 2.4% | 5.7% | 3.4% | 2.5% |
| Step 2 - 2 | 2.4% | 6.0% | 1.3% | 4.6% | 2.6% | 0.0% |

### Example 4—Production of a Fungal Fermentation Medium from Cocoa Shells

Extraction experiments were performed on cocoa shells using eight different conditions as defined in Table 8 below, according to the general protocol as outlined in the section “Thermal extraction of the lignocellulosic material”.

TABLE 8
Summary of the extraction conditions of cocoa shell

| | Treatment Parameters | | |
|---|---|---|---|
| Experiment | Temperature | Time | Severity |
| [—] | [° C.] | [min] | log (Ro) |
| 1 | 150 | 20 | 2.77321468 |
| 2 | 160 | 10 | 2.766621621 |
| 3 | 130 | 30 | 2.360432065 |
| 4 | 140 | 20 | 2.478777743 |
| 5 | 110 | 30 | 1.771558192 |
| 6 | 120 | 20 | 1.889903869 |
| 7 | 130 | 60 | 2.661462061 |
| 8 | 180 | 10 | 3.355495495 |

The data on the recovery of sugars (including C5 sugars) as well as obtainable biomass expressed as per % solid sidestream loaded for comparison purposes (measurements performed as in the general protocols “Analysis of sugar and furfural content” and “Cultivation of fungal biomass” included hereinabove) are summarized in Table 9. In addition, enzymatic hydrolysis of the pretreated lignocellulosic residue obtained after the thermal extraction according to conditions from Table 8 was performed following the protocol “Hydrolysis of the lignocellulosic material” and mycelium was subsequently produced according to the protocol “Cultivation of fungal biomass”. The results of these growth experiments with extracted C6 sugars are also reported in Table 9.

TABLE 9
Summary of the extraction and hydrolysis experiments on cocoa shell

| | Furfural | C5 sugars - | Protein - | Biomass on C5 | | Biomass on C6 |
|---|---|---|---|---|---|---|
| Condition | [g/L per % | Recovery | Recovery | [g/L per % | Complex C5 | [g/L per % |
| (Table 8) | load] | [%] | [%] | load] | [%] | load] |
| 1 | 0 | 26.92035702 | 14.18618444 | 0.7377777778 | n.d. | 0.9688888889 |
| 2 | 0 | 26.1662854 | 17.96916696 | 0.3377777778 | n.d. | 0.56 |
| 3 | 0 | 42.15260386 | 13.71331163 | 0.8511111111 | 100 | 0.8844444444 |
| 4 | 0 | 41.02149642 | 14.65905726 | 0.8266666667 | n.d. | 0.9133333333 |
| 5 | 0 | 27.9760573 | 12.29469318 | 0.8866666667 | n.d. | 1.133333333 |
| 6 | 0 | 29.71042204 | 12.767566 | 0.9088888889 | n.d. | 0.8888888889 |
| 7 | 0 | 43.58533994 | 14.65905726 | 0.9311111111 | n.d. | n.d. |
| 8 | 0.022 | 44.33941157 | 19.38778541 | 0 | 74.49 | n.d. |

Further data on biomass composition obtained from the mycelium grown on the extracts obtained after the thermal treatment, including micronutrients and macronutrients, are shown in Tables 10 and 11, respectively. This data shows clearly that the nature of the lignocellulosic material used for preparing the fermentation medium significantly affect the final composition of the produced mycelium and therefore different lignocellulosic sidestreams will enable the production of different biomasses for the development of different food products.

TABLE 10
Micronutrient composition of biomass grown on cocoa shell
and spent grain C5 extracts (from thermal treatment)

| | Micronutrients | | |
|---|---|---|---|
| | Cocoa shell extract | Standard | |
| Calcium | 1137.679605 | 4386.092876 | mg/kg |
| Potassium | 9221.531203 | 237.2269112 | mg/kg |
| Phospor | 13113.87519 | 1354.937307 | mg/kg |
| Magnesium | 2140.253056 | 1046.85492 | mg/kg |
| Iron | 342.0544714 | 214.3030808 | mg/kg |
| Copper | 41.81857173 | 12.81650516 | mg/kg |
| Zink | 98.97061977 | 88.77774304 | mg/kg |

TABLE 11
Macronutrient composition of biomass grown on cocoa shell
and spent grain C5 extracts (from thermal treatment)

| | Macronutrients | | |
|---|---|---|---|
| | Cocoa shell extract | Standard | |
| Protein | 45.78597469 | 25.91434823 | g/100 g |
| Fat | 9.32875831 | 3.094717099 | g/100 g |
| Ash | 5.586532275 | 2.37574242 | g/100 g |
| Fibers | 24.12609908 | 73.74179431 | g/100 g |

Amino acid composition for the medium obtained from cocoa shells as described herein and the medium obtained from the spent grain, as described in Example 1, are compared in Table 12. It is shown that amino acid composition of the medium may be dependent on the used lignocellulosic material.

TABLE 12
Amino acid composition of thermal extracts
from cocoa shell and spent grain

| | Cocoa | Spent |
|---|---|---|
| | shell | grain |
| Amino | (content | (content |
| acid | in % w/w) | in % w/w) |
| Asp/Asn | 23.40085 | 30.56108 |
| Gln/Glt | 7.401018 | 6.376972 |
| Ser | 3.505641 | 3.518499 |
| His | 0.701289 | 2.039346 |
| Gly | 10.84718 | 10.19775 |
| Thr | 2.431991 | 1.617746 |
| Arg | 6.685 | 4.719055 |
| Ala | 13.83465 | 10.95544 |
| Tyr | 3.866589 | 6.550226 |
| Val | 7.09466 | 3.86828 |
| Met | 5.141458 | 4.409744 |
| Phe | 3.37909 | 4.906722 |
| Ile | 3.215241 | 1.399653 |
| leu | 5.563326 | 3.637575 |
| Lys | 2.932007 | 5.241906 |

### Example 5—Production of a Fungal Fermentation Medium from Olive Cake

Extraction experiments were performed on olive cake using 4 different conditions as defined in Table 13 below, according to the general protocol as outlined in the section “Thermal extraction of the lignocellulosic material”.

TABLE 13
Summary of the extraction conditions of cocoa shell

| | Treatment Parameters | | |
|---|---|---|---|
| Experiment | Temperature | Time | Severity |
| [—] | [° C.] | [min] | log (Ro) |
| 1 | 150 | 20 | 2.773 |
| 2 | 140 | 30 | 2.655 |
| 3 | 160 | 10 | 2.767 |

The data on the recovery of sugars (including C5 sugars) as well as obtainable biomass expressed as per % solid sidestream loaded for comparison purposes are summarized in Table 14. Similarl, to the previous examples, the protocol “Analysis of sugar and furfural content” was used to quantify sugars and furfural and fermentation was performed on the obtained extracts afterwards using the protocol “Cultivation of fungal biomass”. The outcome of both extraction and fermentation are summarized in Table 14. In addition, table 14 also shows the biomass (expressed as per % solid sidestream loaded for comparison purposes) obtained from fermentation on hydrolysates obtained after enzymatic hydrolysis of the thermally pretreated olive cake (Protocol “Hydrolysis of the lignocellulosic material”)

TABLE 14
Summary of the extraction and hydrolysis experiments on olive cake

| | Furfural | C5 sugars - | Protein - | Biomass on C5 | | Biomass on C6 |
|---|---|---|---|---|---|---|
| Condition | [g/L per % | Recovery | Recovery | [g/L per % | Complex C5 | [g/L per % |
| (Table 30) | load] | [%] | [%] | load] | [%] | load] |
| 1 | 0 | 12.8856847 | 15.43373188 | 0.5622222222 | 93.84 | 0.8711111111 |
| 2 | 0 | 9.366901571 | 12.86144324 | 0.5511111111 | n.d. | n.d. |
| 3 | 0 | 13.38128796 | 16.71987621 | 0.5911111111 | 56.00 | 0.94 |

### Example 6—Contamination Test

All the chemicals required for media preparation were purchased from Carl Roth (Karlsruhe, Germany), VWR (Darmstadt, Germany), Merck KgaA (Darmstadt, Germany) or Sigma-Aldrich (Steinheim, Germany).

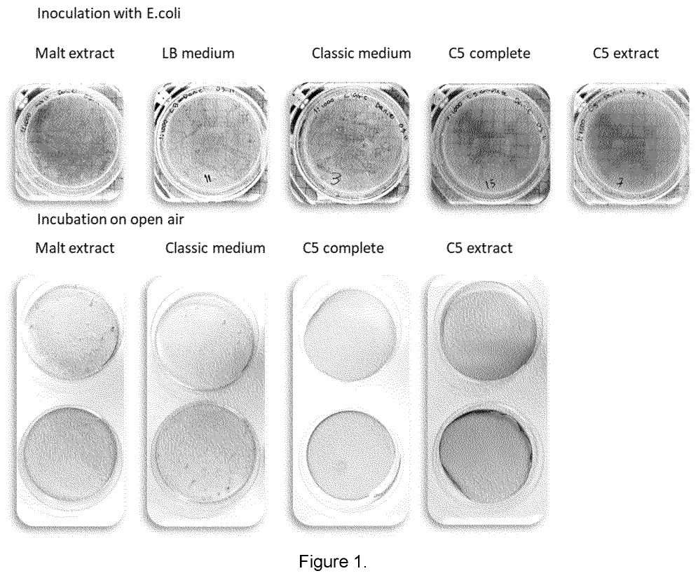

The media for preparation of agar plate were prepared by weighing the different compounds according to Table 15 and dissolving them in water afterwards. The media according to the present invention described as C5 extract were prepared as in Example 1 condition 2. C5 complete refers to a C5 extract as described herein, further supplemented with 5.9 g/I K₂HPO₄, 9.0 g/L KH₂PO₄, 12.0·10⁻² g/L MgSO₄, 8.10·10⁻³ FeCl₃, and 10.10·10-3 CaCl₂. The mixtures were then autoclaved for 20 min at 121° C. und subsequently poured aseptically under a sterile clean bench and let there until complete solidification. Subsequently, the plates prepared with LB medium, malt extract, M9 classic medium, C5 extract and C5 complete were inoculated with a 100 μL 1:1000 diluted E. coli suspension obtained from a culture in LB medium incubated at 37° C. overnight. The plates were then incubated at 37° C. overnight and photographed on the next day. In another experiment, the lid of plates prepared with malt extract, M9 classic medium, C5 extract and C5 complete were just removed and exposed to air contamination to compare growth on the different medium. In that case, pictures of the plates were made after 10 days (see FIG. 1 for summary).

TABLE 15
Composition of media used for the contamination tests

| | |
|---|---|
| Compound | Concentration [g L⁻¹] |

LB medium

| | |
|---|---|
| NaCl | 10 |
| Tryptone | 10 |
| Yeast extract | 5 |
| Agar | 20 |

Fungal fermentation medium

| | |
|---|---|
| Malt extract | 30 |
| Peptone | 5 |
| Agar | 20 |

M9 “Classic medium”

| | |
|---|---|
| Glucose | 10.0 |
| KH₂PO₄ | 3.00 |
| NH₄Cl | 1.50 |
| Na₂HPO₄ | 6.70 |
| FeCl₃ | 8.10 · 10⁻³ |
| CaCl₂ | 10.0 · 10⁻³ |
| MgSO₄ | 12.0 · 10⁻² |
| ZnCl₂ | 17.0 · 10⁻⁴ |
| MnCl₂ | 10.0 · 10⁻⁴ |
| NaMoO₄ · 2H2O | 60.0 · 10⁻⁵ |
| CoCl₂ | 32.8 · 10⁻⁵ |
| CuCl₂ · 2H2O | 43.0 · 10⁻⁵ |
| NaCl | 10.0 |
| Agar | 20.0 |

### Example 7—Production of a Fungal Fermentation Medium from Spent Grain Using Liquid Extraction with Added Acid

Extraction experiments using liquid water were performed on spent grain according to the general protocol described hereinabove, wherein 0.2% or 0.4% w/w of H₂SO₄ were added to the water used for extraction. The experiments were performed according to the conditions as discussed in Table 16.

TABLE 16
Summary of the extraction conditions
of spent grain with diluted acid

Treatment Parameters

| Experiment | Temperature | Time | Severity | H2SO4 |
|---|---|---|---|---|
| [—] | [° C.] | [min] | log (Ro) | [%] |
| 1 | 110 | 20 | 1.595466933 | 0.2 |
| 2 | 110 | 20 | 1.595466933 | 0.4 |
| 3 | 130 | 40 | 2.485370802 | 0.4 |
| 4 | 140 | 30 | 2.654869002 | 0.2 |
| 5 | 140 | 60 | 2.955898998 | 0.4 |
| 6 | 160 | 10 | 2.766621621 | 0.4 |

Similarly to the previous examples, the protocol “Analysis of sugar and furfural content” was used to quantify recovery of C5 sugars and furfural concentration and fermentation was performed on the obtained extracts afterwards using the protocol “Cultivation of fungal biomass”. The outcome of both extraction and fermentation are summarized in Table 17.

TABLE 17
Summary of the extraction experiments
with spent grain using diluted acid

| Condition | Furfural | C5 sugars - | Biomass on C5 |
|---|---|---|---|
| (Table 16) | [g/L per % load] | Recovery [%] | [g/L per % load] |
| 1 | 0.000 | 34.64 | 2.055 |
| 2 | 0.000 | 47.20 | 0.7825 |
| 3 | 0.000 | 72.31 | 1.865 |
| 4 | 0.000 | 62.35 | 1.30 |
| 5 | 0.175 | 96.12 | n.d. |
| 6 | 0.000 | 97.42 | n.d. |

### Example 8: Production of a Fungal Fermentation Medium from Spent Grain Using Steam Prehydrolysis

Extraction experiments with steam prehydrolysis were performed on the spent grain as described in protocol “Thermal pretreatment and extraction of lignocellulosic biomass using steam”. The experimental conditions for the prehydrolysis are listed in Table 18. The results of sugar and furfural analysis (Protocol “Analysis of sugar and furfural content”) as well as the biomass (expressed as per % solid sidestream loaded for comparison purposes) obtained from fermentations with the resulting extracts (Protocol “Cultivation of fungal biomass”) are presented in Table 19.

TABLE 18
Summary of the extraction conditions of
spent grain using steam prehydrolysis

| | Treatment Parameters | | |
|---|---|---|---|
| Experiment | Temperature | Time | Severity |
| [—] | [° C.] | [min] | log (Ro) |
| 1 | 150 | 7.5 | 2.35 |
| 2 | 150 | 15 | 2.65 |
| 3 | 160 | 7.5 | 2.64 |
| 4 | 160 | 15 | 2.94 |
| 5 | 170 | 7.5 | 2.94 |
| 6 | 170 | 15 | 3.24 |
| 7 | 170 | 30 | 3.54 |
| 8 | 180 | 7.5 | 3.23 |
| 9 | 180 | 10 | 3.36 |
| 10 | 180 | 20 | 3.66 |

TABLE 19
Summary of the extraction experiments with
spent grain using steam prehydrolysis

| Condition | C5 sugars - | Biomass on C5 | Complex |
|---|---|---|---|
| (Table 18) | Recovery [%] | [g/L per % load] | C5 [%] |
| 1 | 20.59 | 0.47 | 88.02 |
| 2 | 16.75 | 0.86 | 84.22 |
| 3 | 26.93 | 0.69 | 87.41 |
| 4 | 26.25 | 0.97 | 87.60 |
| 5 | 72.41 | 1.05 | 88.15 |
| 6 | 52.95 | 1.23 | 89.34 |
| 7 | 28.96 | 0.82 | 92.03 |
| 8 | 51.37 | 0.57 | n.d. |
| 9 | 39.83 | 1.34 | n.d. |
| 10 | 36.39 | 0 | n.d. |

### Example 9—Hydrolysis of Cellulose from Pretreated Spent Grain from Example 8

The pretreated solid material left after the steam prehydrolysis and recovery with water was recovered, subjected to a second prehydrolysis and finally to an enzymatic hydrolysis as described in “ ”. The conditions tested are listed in Table 20.

TABLE 20
Summary of the extraction conditions of pretreated
spent grain using steam prehydrolysis

| | Treatment Parameters | | |
|---|---|---|---|
| Experiment | Temperature | Time | Severity |
| [—] | [° C.] | [min] | log (Ro) |
| 1 | 150 | 15 | 2.65 |
| 2 | 160 | 7.5 | 2.64 |
| 3 | 160 | 15 | 2.94 |
| 4 | 170 | 7.5 | 2.94 |
| 5 | 170 | 30 | 3.54 |
| 6 | 170 | 7.5 + 15 | 2.94 + 3.237 |
| 7 | 170 | 7.5 + 30 | 2.94 + 3.538 |
| 8 | 170 | 15 + 30 | 3.237 + 3.538 |

The data on the recovery of C6 sugars as well as obtainable biomass (measurements performed as in the general protocols “Analysis of sugar and furfural content” and “Cultivation of fungal biomass” included hereinabove) are summarized in Table 21. For comparison purposes, they are normalized to the solid load used for the experiments.

TABLE 21
Summary of the extraction experiments with
spent grain using steam prehydrolysis

| Condition | C6 sugars - Recovery | Biomass on C6 |
|---|---|---|
| (Table 20) | [g/L per % load] | [g/L per % load] |
| 1 | 1.66 | n.d. |
| 2 | 1.91 | 1.67 |
| 3 | 2.23 | 1.41 |
| 4 | 1.77 | 1.34 |
| 5 | 2.00 | 1.29 |
| 6 | 2.69 | 1.46 |
| 7 | 2.99 | 1.80 |
| 8 | 3.31 | 1.77 |

### Example 10. Characterization of Meatballs Produced by Using the Biomass of Example 8 in Comparison to Reference Meatballs

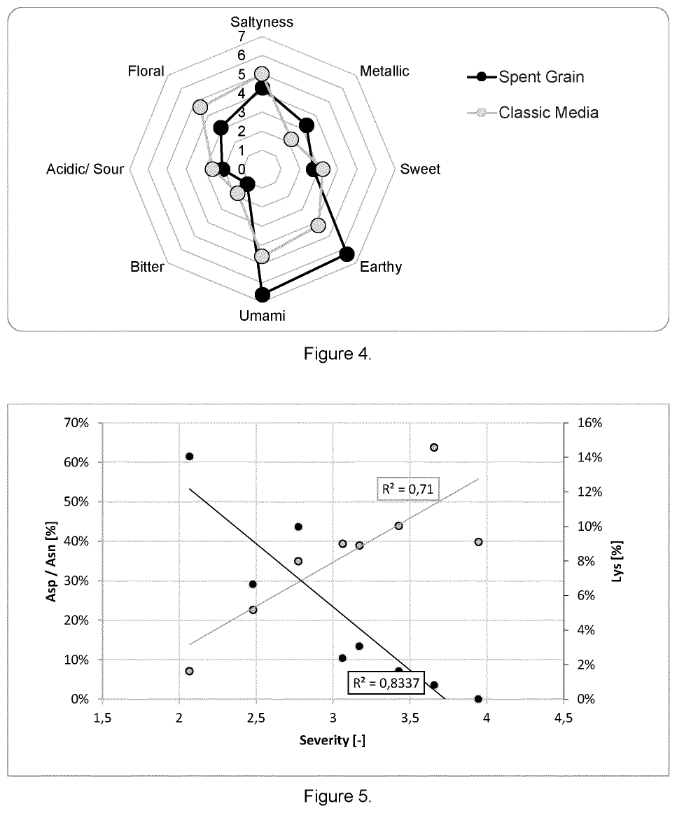

Meatballs were prepared and evaluated as described in protocol “Sensory evaluation of meatballs produced with mycelium from different origins” and the results are summarized in Table 23 and FIG. 4. As can be seen, the meatballs from spent grain were characterised as more umami and earthy (mushroom) as their counterparts from the standard fermentation process and the use of extracts from the side stream shows a significant advantage for the targeted application.

Similarly, the colour and appearance of the obtained meatballs is summarized in Table 23 and FIG. 2 and shows that the meatball grown on the extract from spent grain as a significantly darker colour that is closer to meat than the meatball produced with mycelium from a fermentation with the reference medium. Hence, growing mycelium on extracts from spent grain produced a surprising effect that provides a better taste and appearance for use in meat alternative products. It is expected that similar effects can be expected for sidestreams similar composition and sidestreams with different compositions will certainly produce other surprising effects.

TABLE 22
Characterization of the colour of the meatballs
with mycelium grown on spent grain extract
and mycelium grown on the reference medium

| | Mean | | |
|---|---|---|---|
| | R | G | B |
| Spent grain | 130 | 106.0 | 82.6 |
| Normal | 198.7 | 163.2 | 108.0 |

TABLE 23
Characterization of the sensory profile of the meatballs
produced with mycelium grown on spent grain extract
and mycelium grown on the reference medium

Sensory profile of meatball
with mycelium from

| | Spent Grain | Classic Media |
|---|---|---|
| Saltyness | 4.3 | 5 |
| Metallic | 3.3 | 2.2 |
| Sweet | 2.7 | 3.2 |
| Earthy | 6.3 | 4.2 |
| Umami | 6.6 | 4.6 |
| Bitter | 1.1 | 1.8 |
| Acidic/Sour | 2.1 | 2.6 |
| Floral | 3.1 | 4.6 |
| Off-Flavour | 0.75 | 1.25 |

* Sensory evaluation was performed with 5 panelists

### Example 11: Production of a Fungal Fermentation Medium from Wheat Bran

Extraction experiments were performed on wheat bran using 4 different conditions as defined in Table 24 below, according to the general protocol as outlined in the section “Thermal extraction of the lignocellulosic material”.

TABLE 24
Summary of the extraction conditions of wheat
bran and related fermentation outcome

| | Treatment parameters | | | |
|---|---|---|---|---|
| Experiment | Temperature | Time | Severity | Biomass on C5 extracts |
| [—] | [° C.] | [min] | log (Ro) | [g/L per % load] |
| 1 | 120 | 20 | 1.89 | 2.2632 |
| 2 | 110 | 10 | 1.294 | 0.4555 |
| 3 | 130 | 10 | 1.883 | 1.26775 |
| 4 | 150 | 10 | 2.472 | 0.91725 |

Further embodiments of the invention as disclosed in the following numbered items:

1. A method for the production of a fungal fermentation medium from at least one industrial and/or agricultural side stream, the method comprising:
(a) aqueous extraction of the at least one industrial and/or agricultural side stream; and
(b) combination of the aqueous extract(s) obtained in (a) with optionally at least one nutrient supplement for fungal cultivation.

2. The method according to item 1, wherein (a) is performed with water at a pressure of between 2 and 220 bar and at a temperature of between 9° and 374° C. for a time of between 10 and 200 minutes.

3. The method according to item 1 or 2, wherein (a) is performed with water at a pH of between 2.0 and 12.0, preferably of between 3.0 and 10.0, more preferably of between 4.0 and 8.0, most preferably of between 5.0 and 8.0.

4. The method according to any one of preceding items, further comprising steps of processing the aqueous extract(s) obtained in (a) before the step (b) as follows:
i. proteins are separated from the aqueous extract(s) preferably by flocculation or by precipitation with CO₂;
ii. optionally proteins obtained in i. are hydrolyzed, preferably by using proteolytic enzymes, in particular selected from alcalases, papain, proteinase K, and trypsin, at a concentration of between 0.01% and 5% (w/w) and/or at a temperature of between 15 and 100° C. and/orfor a time of between 0.5 and 96 hours;
iii. C5-polysaccharides present in the product of i. are hydrolyzed optionally using hemicellulases to monosaccharides, in particular xylose and/or arabinose; and
iv. product(s) of steps ii. and/or iii. are further used in step (b).

5. The method according to item 1, wherein (a) involves the steps of:
(a1) extraction of the industrial and/or agricultural side stream with water, optionally supplemented by NaOH at a concentration of between 0.1% to 1.0% w/w, at a temperature of between 90 to 374° C., preferably of between 10° and 220° C., more preferably of between 100 to 180° C., for a time of between 10 to 200 minutes; and
(a2) extraction of the industrial and/or agricultural side stream with water at a temperature of between 12° and 220° C., preferably of between 13° and 200° C. for a time of between 5 and 150 minutes.

6. The method according to item 5, wherein proteins present in the aqueous extract obtained in (a1) are isolated preferably by flocculation or by precipitation with CO₂.

7. The method according to item 5 or 6, further comprising a step wherein proteins present in the aqueous extract obtained in (a1) are hydrolyzed before the step (b).

8. The method according to any one of items 5 to 7 further comprising a step wherein C5-polysaccharides present in the aqueous extract obtained in (a2) are further hydrolyzed to monosaccharides optionally using hemicellulases before the step (b).

9. The method according to item 8, wherein hemicellulases, in particular selected from xylanase, β-glycosidase, α-arabinofuranosidase, α-glucuronidase, and β-xylosidase, are used at a concentration of between 0.01% and 5% (w/w) and/or at a temperature of between 15 and 100° C. and/or for a time of between 0.5 and 96 hours;

10. The method according to any one of preceding items, further comprising the step (a′) of enzymatic hydrolysis of a solid lignocellulosic residue obtained in (a) with cellulase, and separating a liquid product of hydrolysis from a solid residue.

11. The method according to item 10, wherein (a′) is performed at a temperature of between 15 and 100° C. and/or at a pH of between 3.0 and 8.0, and/or for a time of between 10 and 200 hours.

12. The method according to any one of preceding items, wherein the industrial and/or agricultural side stream is a solid side stream, wherein preferably the solid side stream is selected from spent grain, cereal brans, cotton, cotton seed husks, bagasse, cocoa shells, cocoa, cocoa pods, cotton and oil press cakes from sunflower, peanut, hazelnut, palm oil, olive, shells and husks from nuts, grass and leaves waste, wood chips, coffee grounds, coffee husks, coffee silverskin, rapeseed and/or byproducts from the soy industry like soybean pulp (“okara”).

13. The method according to any one of preceding items, further comprising a step of extraction of lipids using supercritical CO₂ and their mechanical separation before the step (a).

14. The method according to any one of preceding items, further comprising a step of removal of toxic compounds present in the aqueous extract of (a) and/or optionally in the liquid product of (a′), such as furfural and/or hydroxymethylfurfural, before step (b).

15. The method according to any one of preceding items, further comprising the step of recovering a solid lignin residue of step (a′).

16. The method according to any one of preceding items, wherein in step b the protein composition obtained according to item 4, 6 or 7 is further supplemented.

17. A protein composition obtained according to item 4, 6 or 7.

18. A fungal fermentation medium obtained in the method according to any one of items 1 to 16.

19. The fungal fermentation medium of item 18, further supplemented with (a) nitrogen source(s), in particular selected from ammonia, urea, yeast extract, malt extract, corn steep liquor and peptone, and or with a carbon source(s), in particular selected from glucose, fructose, sucrose, lactose, maltose, xylose, galactose, dextrose, glycerol, and molasses, and/or with trace elements and/or vitamins.

20. The fungal fermentation medium of item 18 or 19, further processed into a dried form.

21. A method for producing a fungal biomass by submerged fermentation of at least one fungal strain, the method comprising:
(a) providing the pH-adjusted fungal fermentation medium of any one of items 18 to 20 to a fermenter suitable for growing fungal mycelium;
(b) cultivating fungal mycelium; and
(c) retrieving and concentrating the fungal biomass to achieve a dry fungal biomass content of between 2 to 100%.

22. The method according to item 21, wherein step (b) is performed at a temperature of between 15 and 40° C. and/or at a pH of between 3.0 and 8.5 and/or for a time of between 12 and 240 hours.

23. The method according to item 21 or 22, wherein the at least one fungal strain is an edible fungus.

24. The method according to any one of items 21 to 23, wherein the at least one fungal strain is selected from Basidiomycota and Ascomycota.

25. The method according to any one of items 21 to 24, wherein the at least one fungal strain is selected from Pezizomycotina and Agaromycotina.

26. The method according to any one of items 21 to 25, wherein the at least one fungal strain is selected from Peziomycetes, Agaricomycetes and Sordariomycetes

27. The method according to any one of items 21 to 26, wherein the at least one fungal strain is selected from Pezizales, Boletales, Cantharellales, Agaricales, Polyporales, Russulales, Auriculariales, Sordoriales and Hypocreales.

28. The method according to any one of items 21 to 27, wherein the at least one fungal strain is selected from Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, Sordoriaceae, Nectriaceae and Fistulinaceae.

29. The method according to any one of items 21 to 28, wherein the at least one fungal strain is P. pulmonarius P. ostreatus, P. citrinopileatus or P. salmoneostramineus or wherein the at least one fungal strain is M. esculenta, M angusticeps or M. deliciosa.

30. The method according to any one of items 21 to 29, wherein the submerged fermentation is operated as a batch, a fed-batch or a continuous process.

31. The method according to any one of items 21 to 30, wherein more than one fungal strains are co-fermented.

32. A fungal biomass produced according to the method of any one of items 21 to 31.

33. The fungal biomass of item 32, wherein the fungal strain is selected from Pleurotaceae, in particular wherein the fungal strain is P. pulmonarius P. ostreatus, P. citrinopileatus or P. salmoneostramineus, or wherein the at least one fungal strain is selected from Morchellaceae, in particular wherein the fungal strain is M. esculenta, M angusticeps or M. deliciosa.

34. Use of the fungal biomass of item 32 or 33 in production of a fungal-based food product.

35. The use of item 34, wherein the solid lignin residue recovered according to item 15 is further used in production of the fungal-based food product.

36. The use of claim 35, wherein the solid lignin residue recovered according to item 15 is further processed by milling and/or by grinding before being further used in production of the fungal-based food product.

37. The use of any one of items 34 to 36, wherein the protein composition recovered according to item 17 is used in the preparation of the fungal based food product.

38. A fungal-based food product prepared using the fungal biomass of item 32 or 33.

39. The fungal-based food product of item 38, wherein the solid lignin residue recovered according to item 15 is used in the preparation of the fungal-based food product.

40. The fungal-based food product of item 38 or 39, wherein the protein composition recovered according to item 17 is used in the preparation of the fungal-based food product.

Further embodiments of the invention as disclosed in the following numbered paragraphs:

1. A method for the production of a fungal fermentation medium from at least one industrial and/or agricultural side stream, the method comprising:
(a) aqueous extraction of the at least one industrial and/or agricultural side stream, and
(b) combination of the aqueous extract(s) obtained in (a) with optionally at least one nutrient supplement for fungal cultivation.

2. The method according to paragraph 1, wherein (a) is performed with water at a pressure of between 2 and 220 bar and at a temperature of between 9° and 374° C. for a time of between 10 and 200 minutes,
and/or
wherein (a) is performed with water at a pH of between 2.0 and 12.0, preferably of between 3.0 and 10.0, more preferably of between 4.0 and 8.0, most preferably of between 5.0 and 8.0.

3. The method according to any one of preceding paragraphs, further comprising steps of processing the aqueous extract(s) obtained in (a) before the step (b) as follows:
i. proteins are separated from the aqueous extract(s) preferably by flocculation or by precipitation with CO₂;
ii. optionally proteins obtained in i. are hydrolyzed, preferably by using proteolytic enzymes, in particular selected from alcalases, papain, proteinase K, and trypsin, at a concentration of between 0.01% and 5% (w/w) and/or at a temperature of between 15 and 100° C. and/or for a time of between 0.5 and 96 hours;
iii. C5-polysaccharides present in the product of i. are hydrolyzed optionally using hemicellulases to monosaccharides, in particular xylose and/or arabinose; and
iv. product(s) of steps ii. and/or iii. are further used in step (b).

4. The method according to paragraph 1, wherein (a) involves the steps of:
(a1) extraction of the industrial and/or agricultural side stream with water, optionally supplemented by NaOH at a concentration of between 0.1% to 1.0% w/w, at a temperature of between 90 to 374° C., preferably of between 10° and 220° C., more preferably of between 100 to 180° C., for a time of between 10 to 200 minutes; and
(a2) extraction of the industrial and/or agricultural side stream with water at a temperature of between 12° and 220° C., preferably of between 13° and 200° C., for a time of between 5 and 150 minutes,
preferably wherein proteins present in the aqueous extract obtained in (a1) are isolated preferably by flocculation or by precipitation with CO₂, optionally further comprising a step wherein proteins present in the aqueous extract obtained in (a1) are hydrolyzed before the step (b), optionally further comprising a step wherein C5-polysaccharides present in the aqueous extract obtained in (a2) are further hydrolyzed to monosaccharides optionally using hemicellulases before the step (b), preferably wherein hemicellulases, in particular selected from xylanase, β-glycosidase, α-arabinofuranosidase, α-glucuronidase, and β-xylosidase, are used at a concentration of between 0.01% and 5% (w/w) and/or at a temperature of between 15 and 100° C. and/or for a time of between 0.5 and 96 hours;

5. The method according to any one of preceding paragraphs, further comprising the step (a′) of enzymatic hydrolysis of a solid lignocellulosic residue obtained in (a) with cellulase, and separating a liquid product of hydrolysis from a solid residue, preferably wherein (a′) is performed at a temperature of between 15 and 100° C. and/or at a pH of between 3.0 and 8.0, and/or for a time of between 10 and 200 hours.

6. The method according to any one of preceding paragraphs, wherein the industrial and/or agricultural side stream is a solid side stream, wherein preferably the solid side stream is selected from spent grain, cereal brans, cotton, cotton seed husks, bagasse, cocoa shells, cocoa, cocoa pods, cotton and oil press cakes from sunflower, peanut, hazelnut, palm oil, olive, shells and husks from nuts, grass and leaves waste, wood chips, coffee grounds, coffee husks, coffee silverskin, rapeseed and/or byproducts from the soy industry like soybean pulp (“okara”).

7. The method according to any one of preceding paragraphs, further comprising a step of extraction of lipids using supercritical CO₂ and their mechanical separation before the step (a),
and/or
further comprising a step of removal of toxic compounds present in the aqueous extract of (a) and/or optionally in the liquid product of (a′), such as furfural and/or hydroxymethylfurfural, before step (b),
and/or
further comprising the step of recovering a solid lignin residue of step (a′).

8. A protein composition obtained according to paragraph 3 or 4.

9. A fungal fermentation medium obtained in the method according to any one of paragraphs 1 to 7, optionally further supplemented with (a) nitrogen source(s), in particular selected from ammonia, urea, yeast extract, malt extract, corn steep liquor and peptone, and or with a carbon source(s), in particular selected from glucose, fructose, sucrose, lactose, maltose, xylose, galactose, dextrose, glycerol, and molasses, and/or with trace elements and/or vitamins, optionally further processed into a dried form.

10. A method for producing a fungal biomass by submerged fermentation of at least one fungal strain, the method comprising:
(a) providing the pH-adjusted fungal fermentation medium of claim 9 to a fermenter suitable for growing fungal mycelium;
(b) cultivating fungal mycelium; and
(c) retrieving and concentrating the fungal biomass to achieve a dry fungal biomass content of between 2 to 100%, preferably wherein step (b) is performed at a temperature of between 15 and 40° C. and/or at a pH of between 3.0 and 8.5 and/or for a time of between 12 and 240 hours.

11. The method according to paragraph 10, wherein the at least one fungal strain is an edible fungus, preferably wherein the at least one fungal strain is selected from Basidiomycota and Ascomycota, more preferably wherein the at least one fungal strain is selected from Pezizomycotina and Agaromycotina, even more preferably wherein the at least one fungal strain is selected from Peziomycetes, Agaricomycetes and Sordariomycetes, even more preferably wherein the at least one fungal strain is selected from Pezizales, Boletales, Cantharellales, Agaricales, Polyporales, Russulales, Auriculariales, Sordoriales and Hypocreales, even more preferably wherein the at least one fungal strain is selected from Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, Sordoriaceae, Nectriaceae and Fistulinaceae, most preferably wherein the at least one fungal strain is P. pulmonarius P. ostreatus, P. citrinopileatus or P. salmoneostramineus or wherein the at least one fungal strain is M. esculenta, M angusticeps or M. deliciosa.

12. The method according to paragraph 10 or 11, wherein the submerged fermentation is operated as a batch, a fed-batch or a continuous process,
and/or
wherein more than one fungal strains are co-fermented.

13. A fungal biomass produced according to the method of any one of paragraphs 10 to 12, preferably wherein the fungal strain is selected from Pleurotaceae, in particular wherein the fungal strain is P. pulmonarius P. ostreatus, P. citrinopileatus or P. salmoneostramineus, or wherein the at least one fungal strain is selected from Morchellaceae, in particular wherein the fungal strain is M. esculenta, M angusticeps or M. deliciosa.

14. Use of the fungal biomass of paragraph 13 in production of a fungal-based food product.

15. A fungal-based food product prepared using the fungal biomass of paragraph 13.

1. A method for the production of a fungal fermentation medium from at least one lignocellulosic material comprising at least one industrial and/or agricultural side stream, the method comprising:
(a) extracting an aqueous extract(s) from the at least one industrial and/or agricultural side stream; and
(b) optionally combining the aqueous extract(s) obtained in step (a) with at least one nutrient supplement for fungal cultivation.

2. The method according to claim 1, wherein the step (a) includes a washing step performed with liquid water at the temperature of 50° C. to 100° C.

3. The method according to claim 1, wherein during step (a) lignocellulosic materials are contacted with steam at a temperature of between 130° C. and 180° C., for a time of up to 20 minutes.

4. The method according to claim 3, wherein the lignocellulosic material subject to prehydrolysis with steam followed by washing is washed with liquid water at the temperature of 50° C. to 100° C.

5. The method according to claim 1, wherein (a) is performed with water at a pressure of between 1.25 and 220 bar and at a temperature of between 90° and 374° C. for a time of between 10 and 200 minutes,
and/or
wherein (a) is performed with water at a pH of between 2.0 and 12.0.

6. The method according to claim 1, further comprising steps of processing the aqueous extract(s) obtained in (a) before the step (b) as follows:
(i) separating proteins from the aqueous extract(s) by flocculation or by precipitation with CO₂;
(ii) hydrolyzing proteins obtained in step (i) by using proteolytic enzymes at a concentration of between 0.01% and 5% (w/w) and/or at a temperature of between 15 and 100° C. and/or for a time of between 0.5 and 96 hours; and
(iii) hydrolyzing C5-polysaccharides present in the product of step (i) to monosaccharides; and
(iv) wherein product(s) of step (ii) and/or step (iii) are further used in step (b).

7. (canceled)

8. The method according to claim 1, further comprising a step (a′) comprising enzymatically hydrolyzing a solid lignocellulosic residue obtained in step (a) with cellulase, and separating a liquid product of hydrolysis from a solid residue.

9-10. (canceled)

11. The method according to claim 1, wherein the industrial and/or agricultural side stream is a solid side stream selected from spent grain, cereal brans, cotton, cotton seed husks, bagasse, cocoa shells, cocoa, cocoa pods, cotton and oil press cakes from sunflower, peanut, hazelnut, palm oil, olive, shells and husks from nuts, grass and leaves waste, wood chips, coffee grounds, coffee husks, coffee silverskin, rapeseed, and/or byproducts from the soy industry like soybean pulp (“okara).

12. (canceled)

13. The method according to claim 8, further comprising a step of extraction of lipids using supercritical CO₂ and their mechanical separation before the step (a),
and/or
further comprising a step of removal of toxic compounds present in the aqueous extract of step (a),
and/or
further comprising the step of recovering a solid lignin residue of step (a′).

14. A protein composition obtainable by the method according to claim 6.

15. A fungal fermentation medium obtainable in by the method according to claim 1.

16. The fungal fermentation medium of claim 15, wherein said medium is further supplemented with nitrogen source(s).

17. The fungal fermentation medium of claim 16, wherein said medium is further processed into a dried form.

18. The fungal fermentation medium of claim 15, wherein C5-polysaccharides constitute at least 50% of all sugars in said medium.

19. A method for producing a fungal biomass by submerged fermentation of at least one fungal strain, the method comprising:
(a) providing the pH-adjusted fungal fermentation medium of claim 15 to a fermenter suitable for growing fungal mycelium;
(b) cultivating fungal mycelium; and
(c) retrieving and concentrating the fungal biomass to achieve a dry fungal biomass content of between 2 to 100%.

20. The method according to claim 19, wherein the at least one fungal strain is an edible fungus.

21-24. (canceled)

25. The method according to claim 19, wherein the at least one fungal strain is selected from Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, Sordoriaceae, Nectriaceae, Fistulinaceae, and combinations thereof.

26. The method according to claim 19, wherein the at least one fungal strain is P. pulmonarius P. ostreatus, P. citrinopileatus or P. salmoneostramineus or wherein the at least one fungal strain is M. esculenta, M angusticeps or M. deliciosa.

27-29. (canceled)

30. A fungal biomass produced according to the method of claim 19.

31. (canceled)

32. A method of producing a fungal-based food product using the fungal biomass produced according to the method of claim 1.

33-34. (canceled)

35. A fungal-based food product prepared using the fungal biomass of claim 30.

36. The method according to claim 1, wherein the aqueous extract(s) is extracted from the at least one industrial and/or agricultural side stream with an aqueous solution comprising 1% w/w or less of acid or base in step (a).

37. The method according to claim 5, wherein the aqueous extract(s) is extracted from the at least one industrial and/or agricultural side stream with an aqueous solution comprising 1% w/w or less of acid or base in step (a).
